(19) United States
(12) Patent Application Publication
(10) Pub. No.: US 2003/0175806 A1
(43) Pub. Date: Sep. 18, 2003

(54) Method and apparatus for improving the accuracy of alternative site analyte concentration measurements

(76) Inventors: Peter Rule, Los Altos Hills, CA (US); James R. Braig, Piedmont, CA (US); Phillip C. Hartstein, Cupertino, CA (US); Heidi S. Smith, Union City, CA (US); David C. Klonoff, Burlingame, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/302,071

(22) Filed: Nov. 21, 2002

Related U.S. Application Data
(60) Provisional application No. 60/332,093, filed on Nov. 21, 2001.
(60) Provisional application No. 60/354,436, filed on Feb. 4, 2002.

(51) Int. Cl. G01N 33/53; G06F 19/00; G01N 33/48; G01N 33/50

(52) U.S. Cl. 435/7.1; 702/19

(57) ABSTRACT

Methods and apparatuses for improving the accuracy of analyte concentration measurements at alternative sites and information provided to the user. The present invention advantageously utilizes information relating to the rate of change of analyte concentrations to adjust analyte concentrations measurements and/or information provided to the user. Therefore, the present invention provides new and improved methods and apparatuses for obtaining analyte concentration information at physiological sites other than the fingertips while ensuring that the analyte concentration information accurately reflects systemic analyte concentration values.

### RELATED APPLICATIONS

This application claims the benefit of U.S. Provisional Application No. 60/332,093, filed Nov. 21, 2001, and of U.S. Provisional Application No. 60/354,436, filed Feb. 4, 2002; the disclosures of the aforementioned applications are hereby incorporated in their entirety herein by reference.

### BACKGROUND OF THE INVENTION

1. Field of the Invention

This present invention relates generally to determining analyte concentrations in material samples. More specifically, the present invention relates to methods and apparatuses for improving the accuracy of blood-constituent measurements and/or blood-constituent measurement devices.

2. Description of the Related Art

The analysis of materials and the determination of the presence or concentration of chemical species contained therein is a common and important process in chemistry, biology, and the medical sciences. Particularly important is the analysis of biological fluids, such as, for example, blood, urine, or saliva, to determine the concentration of various constituents. Also of great importance is the measurement of the concentration of various chemical constituents embedded within biological materials, such as, for example, tissue. Chemical analysis of blood, urine, and other biological fluids is crucial to the diagnosis, management, treatment, and care of a wide variety of diseases and medical conditions.

Analysis of various blood components is of importance in both the diagnosis and treatment of diseases of the circulatory system. For example, the level of various types of cholesterol in the blood has a strong correlation with the onset of heart disease. Urine analysis provides valuable information relating to kidney function and kidney disease. The concentration of alcohol in the blood is known to be related to a subject's physical response time and coordination and can provide information related to, for example, the individual's fitness to drive a motor vehicle. In the case of diabetes, accurate monitoring of blood glucose levels is necessary for efficient management of this disease.

Millions of diabetics draw samples of bodily fluid such as blood on a daily basis to monitor the level of glucose in their bloodstream. Traditional blood samples for Self Monitoring of Blood Glucose (“SMBG”) are taken from a finger with a lance. Tests have shown that finger blood glucose levels correlate highly to systemic blood glucose values. Fingertips are highly vaseularized and therefore offer an ideal site from which to draw relatively large (1-3 uL) drops of blood for SMBG analysis. Fingertips are also highly innervated, providing good tactile sense which unfortunately makes lancing to obtain the blood sample painful. What is needed is a method and apparatus for obtaining accurate blood glucose measurements at an alternative site (i.e. a site other than at the fingertips).

### SUMMARY OF THE INVENTION

In accordance with one embodiment, there is provided a method of improving the accuracy of analyte concentration measurements at alternative sites. The method disclosed herein involves: determining a rate of change of analyte concentration; measuring an analyte concentration at an alternative site; and adjusting the measured analyte concentration based on the rate of change to generate an adjusted analyte concentration value which is a more accurate estimate of the systemic analyte concentration value.

In accordance with another embodiment, there is provided a method for improving the accuracy of information provided to a user by an analyte detection system. This method involves determining a rate of change of analyte concentration and adjusting the information provided to the user based on the rate of change.

In accordance with yet another embodiment, there is provided a system for measuring the concentration of an analyte at an alternative site which has a processing circuit and a module executable by the processing circuit whereby the processing circuit receives an analyte concentration measurement taken at the alternative site, determines a rate of change of the concentration of the analyte, and provides information to the user based on the rate of change.

In accordance with a further embodiment, there is provided a system for measuring the concentration of an analyte at an alternative site which has a processing circuit and a module executable by the processing circuit whereby the processing circuit receives an analyte concentration measurement taken at the alternative site, determines a rate of change of the concentration of the analyte, and adjusts said analyte concentration measurement based on said rate of change.

Another embodiment involves determining the clinical significance of an error in the measured analyte concentration.

Another embodiment involves adjusting the measured analyte concentration by: determining a time lag between analyte concentrations measured on-site and at an alternative site; determining a correction factor based on the time lag and the rate of change of analyte concentration; and adjusting the measured analyte concentration value by the correction factor.

Another embodiment involves correcting the measured analyte concentration based on the rate of change of the analyte concentration. Here, correction of the measured analyte concentration involves determining the time lag between analyte concentrations measured on-site and at an alternative site. Correction also involves taking a follow-up analyte concentration measurement at a time that falls between the time of previous analyte concentration measurement and the sum of the time lag and the time of the previous analyte concentration measurement. Correction also involves determining the rate of analyte concentration change during the time period between the previous analyte concentration measurement and the follow-up analyte concentration measurement. Correction also involves extrapolating the analyte concentration value to a time after the sum of the time lag and the time of the previous analyte concentration measurement by using the rate of analyte concentration change in between the previous analyte concentration measurement and the follow-up analyte concentration measurement.

Another embodiment involves screening measured analyte concentration values to remove outlier concentration values and/or rate of concentration change values based on statistical analysis, data history, and/or data forecasts or trends.

Another embodiment relates to the determination of the rate of change of analyte concentration by: estimating that the absolute value of the rate of change is relatively high; estimating that the absolute value of the rate of change will remain relatively high for about 1-2 hours; and/or receiving data input from the user and estimating, based on the data, that the absolute value of the rate of change is relatively high.

Another embodiment relates to the determination of the rate of change of analyte concentration by: measuring a first analyte concentration at a first measurement time; measuring a second analyte concentration at a second measurement time; and computing the rate of change based on the first and second analyte concentrations and the first and second measurement times.

Another embodiment relates to adjusting information provided to the user by: measuring a raw analyte concentration; calculating a corrected analyte concentration based on the rate of change and the raw analyte concentration; assessing the clinical significance of a measurement error based on the raw analyte concentration and the corrected, analyte concentration; and/or warning the user to take an on-site analyte concentration measurement based on the clinical significance of the measurement error.

Another embodiment relates to providing information to the user by: warning the user that the absolute value of the rate of change is high; prompting the user to take an on-site analyte concentration measurement; warning the user, when the absolute value of said rate of change is relatively high, that alternative-site measurements are likely to be inaccurate; prompting the user to take an on-site analyte concentration measurement; and/or warning the user that the concentration of the analyte is increasing or decreasing.

Another embodiment involves a timing circuit accessible by the processing circuit of the system that measures the concentration of an analyte at an alternative site.

Another embodiment involves a system with a user-interface whereby the user is able to input data into the analyte detection system, wherein the processing circuit considers said data in adjusting the analyte concentration to generate an adjusted analyte concentration value.

### BRIEF DESCRIPTION OF THE DRAWINGS

FIG. 1 is a schematic view of a noninvasive optical detection system.

FIG. 2 is a perspective view of a window assembly for use with the noninvasive detection system.

FIG. 2A is a plan view of another embodiment of a window assembly for use with the noninvasive detection system.

FIG. 3 is an exploded schematic view of another embodiment of a window assembly for use with the noninvasive detection system.

FIG. 4 is a plan view of the window assembly connected to a cooling system.

FIG. 5 is a plan view of the window assembly connected to a cold reservoir.

FIG. 6 is a cutaway view of a heat sink for use with the noninvasive detection system.

FIG. 6A is a cutaway perspective view of a lower portion of the noninvasive detection system of FIG. 1.

FIG. 6B is an exploded perspective view of a window mounting system for use with the noninvasive optical detection system.

FIG. 6C is a partial plan view of the window mounting system of FIG. 6B.

FIG. 6D is a sectional view of the window mounting system of FIG. 6C.

FIG. 7 is a schematic view of a control system for use with the noninvasive optical detection system.

FIG. 8 depicts a first methodology for determining the concentration of an analyte of interest.

FIG. 9 depicts a second methodology for determining the concentration of an analyte of interest.

FIG. 10 depicts a third methodology for determining the concentration of an analyte of interest.

FIG. 11 depicts a fourth methodology for determining the concentration of an analyte of interest.

FIG. 12 depicts a fifth methodology for determining the concentration of an analyte of interest.

FIG. 13 is a schematic view of a reagentless whole-blood detection system.

FIG. 14 is a perspective view of one embodiment of a cuvette for use with the reagentless whole-blood detection system.

FIG. 15 is a plan view of another embodiment of a cuvette for use with the reagentless whole-blood detection system.

FIG. 16 is a disassembled plan view of the cuvette shown in FIG. 15.

FIG. 16A is an exploded perspective view of the cuvette of FIG. 15.

FIG. 17 is a side view of the cuvette of FIG. 15.

FIG. 18 is a perspective view of an AST device with meal and exercise buttons.

FIG. 19 depicts the classification of erroneous measurements by their clinical implications.

FIG. 20 depicts a methodology for determining the corrector factor for an AST device user.

FIG. 21 depicts the determination of AST time lag specific to an individual.

### DETAILED DESCRIPTION OF THE PREFERRED EMBODIMENTS

The highly innervated nature of the fingertips makes it desirable patients to take blood glucose measurements at an alternative site. Alternative site (“AST”) testing can be conducted at sites less highly innervated. One of the most popular alternative sites is the forearm. However, the concentrations of blood constituents such as glucose measured at alternative sites, particularly the forearm, can exhibit a time lag relative to simultaneous concentration measurements taken at the finger.

The time lag associated with certain alternative site measurements can be minimized or corrected for by utilizing the rate of change of analyte concentration in the measurement device and/or the time lag between analyte concentrations measured on-site and at an alternative site. Disclosed here are methods and apparatus for methods and apparatuses for improving the accuracy of an AST blood-constituent measurement device. Part I describes the measurement of an analyte by using systems such as a non-invasive analyte detection system or a whole blood analyte detection system. In a presently preferred embodiment, the analyte concentration measurement system measures the concentration of glucose in blood. An analyte concentration measurement is clinically accurate when the measurement results in correct treatment of a patient. However, an instrument may erroneously report a measurement that causes a patient to receive the wrong treatment. Part II describes a method and apparatus for improving the accuracy of alternative site analyte concentration measurements. In a presently preferred embodiment, the rate of analyte concentration change and/or the time lag associated with the patient are utilized to provide accurate analyte concentration measurements at the AST.

Although certain preferred embodiments and examples are disclosed below, it will be understood by those skilled in the art that the invention extends beyond the specifically disclosed embodiments to other alternative embodiments and/or uses of the invention and obvious modifications and equivalents thereof. Thus, it is intended that the scope of the invention herein disclosed should not be limited by the particular disclosed embodiments described below.

### Overview of Analyte Detection Systems

Disclosed herein are analyte detection systems, including a noninvasive system discussed largely in part A below and a whole-blood system discussed largely in part B below. Also disclosed are various methods, including methods for detecting the concentration of an analyte in a material sample. Both the noninvasive system/method and the whole-blood system/method can employ optical measurement. As used herein with reference to measurement apparatus and methods, “optical” is a broad term and is used in its ordinary sense and refers, without limitation, to identification of the presence or concentration of an analyte in a material sample without requiring a chemical reaction to take place. As discussed in more detail below, the two approaches each can operate independently to perform an optical analysis of a material sample. The two approaches can also be combined in an apparatus, or the two approaches can be used together to perform different steps of a method.

In one embodiment, the two approaches are combined to perform calibration of an apparatus, e.g., of an apparatus that employs a noninvasive approach. In another embodiment, an advantageous combination of the two approaches performs an invasive measurement to achieve greater accuracy and a whole-blood measurement to minimize discomfort to the patient. For example, the whole-blood technique may be more accurate than the noninvasive technique at certain times of the day, e.g., at certain times after a meal has been consumed, or after a drug has been administered.

It should be understood, however, that any of the disclosed devices may be operated in accordance with any suitable detection methodology, and that any disclosed method may be employed in the operation of any suitable device. Furthermore, the disclosed devices and methods are applicable in a wide variety of situations or modes of operation, including but not limited to invasive, noninvasive, intermittent or continuous measurement, subcutaneous implantation, wearable detection systems, or any combination thereof.

Any method which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the method(s) in question.

A. Noninvasive System

1. Monitor Structure

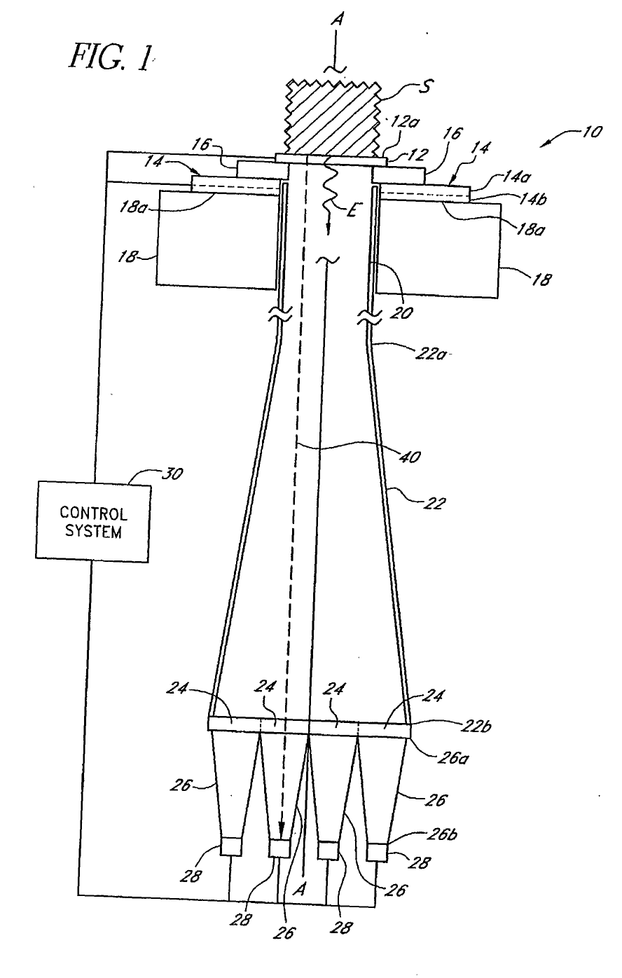

FIG. 1 depicts a noninvasive optical detection system (hereinafter “noninvasive system”) 10 in a presently preferred configuration. The depicted noninvasive system 10 is particularly suited for noninvasively detecting the concentration of an analyte in a material sample S, by observing the infrared energy emitted by the sample, as will be discussed in further detail below.

As used herein, the term “noninvasive” is a broad term and is used in its ordinary sense and refers, without limitation, to analyte detection devices and methods which have the capability to determine the concentration of an analyte in in-vivo tissue samples or bodily fluids. It should be understood, however, that the noninvasive system 10 disclosed herein is not limited to noninvasive use, as the noninvasive system 10 may be employed to analyze an in-vitro fluid or tissue sample which has been obtained invasively or noninvasively. As used herein, the term “invasive” (or, alternatively, “traditional”) is a broad term and is used in its ordinary sense and refers, without limitation, to analyte detection methods which involve the removal of fluid samples through the skin. As used herein, the term “material sample” is a broad term and is used in its ordinary sense and refers, without limitation, to any collection of material which is suitable for analysis by the noninvasive system 10. For example, the material sample S may comprise a tissue sample, such as a human forearm, placed against the noninvasive system 10. The material sample S may also comprise a volume of a bodily fluid, such as whole blood, blood component(s), interstitial fluid or intercellular fluid obtained invasively, or saliva or urine obtained noninvasively, or any collection of organic or inorganic material. As used herein, the term “analyte” is a broad term and is used in its ordinary sense and refers, without limitation, to any chemical species the presence or concentration of which is sought in the material sample S by the noninvasive system 10. For example, the analyte(s) which may be detected by the noninvasive system 10 include but not are limited to glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, electrolytes, sodium, potassium, chloride, bicarbonate, and hormones. As used herein to describe measurement techniques, the term “continuous” is a broad term and is used in its ordinary sense and refers, without limitation, to the taking of discrete measurements more frequently than about once every 10 minutes, and/or the taking of a stream or series of measurements or other data over any suitable time interval, for example, over an interval of one to several seconds, minutes, hours, days, or longer. As used herein to describe measurement techniques, the term “intermittent” is a broad term and is used in its ordinary sense and refers, without limitation, to the taking of measurements less frequently than about once every 10 minutes.

The noninvasive system 10 preferably comprises a window assembly 12, although in some embodiments the window assembly 12 may be omitted. One function of the window assembly 12 is to permit infrared energy E to enter the noninvasive system 10 from the sample S when it is placed against an upper surface 12a of the window assembly 12. The window assembly 12 includes a heater layer (see discussion below) which is employed to heat the material sample S and stimulate emission of infrared energy therefrom. A cooling system 14, preferably comprising a Peltier-type thermoelectric device, is in thermally conductive relation to the window assembly 12 so that the temperature of the window assembly 12 and the material sample S can be manipulated in accordance with a detection methodology discussed in greater detail below. The cooling system 14 includes a cold surface 14a which is in thermally conductive relation to a cold reservoir 16 and the window assembly 12, and a hot surface 14b which is in thermally conductive relation to a heat sink 18.

As the infrared energy E enters the noninvasive system 10, it first passes through the window assembly 12, then through an optical mixer 20, and then through a collimator 22. The optical mixer 20 preferably comprises a light pipe having highly reflective inner surfaces which randomize the directionality of the infrared energy E as it passes therethrough and reflects against the mixer walls. The collimator 22 also comprises a light pipe having highly-reflective inner walls, but the walls diverge as they extend away from the mixer 20. The divergent walls cause the infrared energy E to tend to straighten as it advances toward the wider end of the collimator 22, due to the angle of incidence of the infrared energy when reflecting against the collimator walls.

From the collimator 22 the infrared energy E passes through an array of filters 24, each of which allows only a selected wavelength or band of wavelengths to pass therethrough. These wavelengths/bands are selected to highlight or isolate the absorptive effects of the analyte of interest in the detection methodology discussed in greater detail below. Each filter 24 is preferably in optical communication with a concentrator 26 and an infrared detector 28. The concentrators 26 have highly reflective, converging inner walls which concentrate the infrared energy as it advances toward the detectors 28, increasing the density of the energy incident upon the detectors 28.

The detectors 28 are in electrical communication with a control system 30 which receives electrical signals from the detectors 28 and computes the concentration of the analyte in the sample S. The control system 30 is also in electrical communication with the window 12 and cooling system 14, so as to monitor the temperature of the window 12 and/or cooling system 14 and control the delivery of electrical power to the window 12 and cooling system 14.

2. Window Assembly

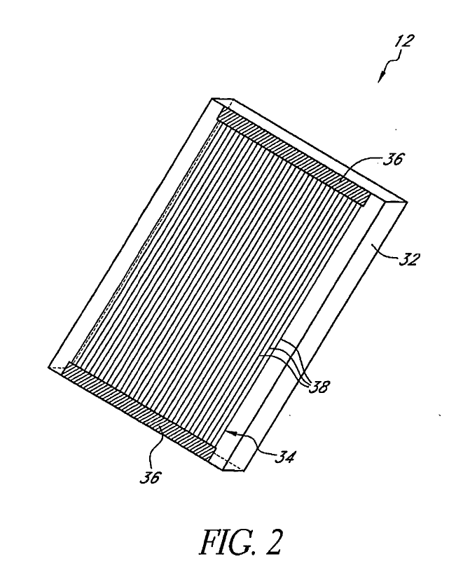

A preferred configuration of the window assembly 12 is shown in perspective, as viewed from its underside (in other words, the side of the window assembly 12 opposite the sample S), in FIG. 2. The window assembly 12 generally comprises a main layer 32 formed of a highly infrared-transmissive material and a heater layer 34 affixed to the underside of the main layer 32. The main layer 32 is preferably formed from diamond, most preferably from chemical-vapor-deposited (“CVD”) diamond, with a preferred thickness of about 0.25 millimeters. In other embodiments alternative materials which are highly infrared-transmissive, such as silicon or germanium, may be used in forming the main layer 32.

The heater layer 34 preferably comprises bus bars 36 located at opposing ends of an array of heater elements 38. The bus bars 36 are in electrical communication with the elements 38 so that, upon connection of the bus bars 36 to a suitable electrical power source (not shown) a current may be passed through the elements 38 to generate heat in the window assembly 12. The heater layer 34 may also include one or more temperature sensors (not shown), such as thermistors or resistance temperature devices (RTDs), to measure the temperature of the window assembly 12 and provide temperature feedback to the control system 30 (see FIG. 1).

Still referring to FIG. 2, the heater layer 34 preferably comprises a first adhesion layer of gold or platinum (hereinafter referred to as the “gold” layer) deposited over an alloy layer which is applied to the main layer 32. The alloy layer comprises a material suitable for implementation of the heater layer 34, such as, by way of example, 10/90 titanium/tungsten, titanium/platinum, nickel/chromium, or other similar material. The gold layer preferably has a thickness of about 4000 Å, and the alloy layer preferably has a thickness ranging between about 300 Å and about 500 Å. The gold layer and/or the alloy layer may be deposited onto the main layer 32 by chemical deposition including, but not necessarily limited to, vapor deposition, liquid deposition, plating, laminating, casting, sintering, or other forming or deposition methodologies well known to those or ordinary skill in the art. If desired, the heater layer 34 may be covered with an electrically insulating coating which also enhances adhesion to the main layer 32. One preferred coating material is aluminum oxide. Other acceptable materials include, but are not limited to, titanium dioxide or zinc selenide.

The heater layer 34 may incorporate a variable pitch distance between centerlines of adjacent heater elements 38 to maintain a constant power density, and promote a uniform temperature, across the entire layer 34. Where a constant pitch distance is employed, the preferred distance is at least about 50-100 microns. Although the heater elements 38 generally have a preferred width of about 25 microns, their width may also be varied as needed for the same reasons stated above.

Alternative structures suitable for use as the heater layer 34 include, but are not limited to, thermoelectric heaters, radiofrequency (RF) heaters, infrared radiation heaters, optical heaters, heat exchangers, electrical resistance heating grids, wire bridge heating grids, or laser heaters. Whichever type of heater layer is employed, it is preferred that the heater layer obscures about 10% or less of the window assembly 12.

In a preferred embodiment, the window assembly 12 comprises substantially only the main layer 32 and the heater layer 34. Thus, when installed in an optical detection system such as the noninvasive system 10 shown in FIG. 1, the window assembly 12 will facilitate a minimally obstructed optical path between a (preferably flat) upper surface 12a of the window assembly 12 and the infrared detectors 28 of the noninvasive system 10. The optical path 32 in the preferred noninvasive system 10 proceeds only through the main layer 32 and heater layer 34 of the window assembly 12 (including any antireflective, index-matching, electrical insulating or protective coatings applied thereto or placed therein), through the optical mixer 20 and collimator 22 and to the detectors 28.

FIG. 2a shows another embodiment of the window assembly 12, that may be used in place of the window assembly 12 depicted in FIG. 2. The window assembly 12 shown in FIG. 2A may be similar to that shown in FIG. 2, except as described below. In the embodiment of FIG. 2A the main layer 32 has a preferred thickness of up to about 0.012″ and more preferably about 0.010″ or less. The heater layer 34 may also include one or more resistance temperature devices (RTD's) 55 to measure the temperature of the window assembly 12 and provide temperature feedback to a control system 30. The RTDs 55 terminate in RTD connection pads 57.

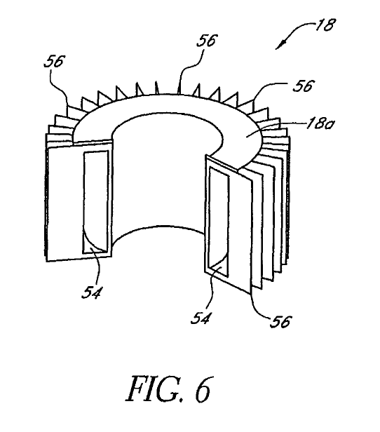

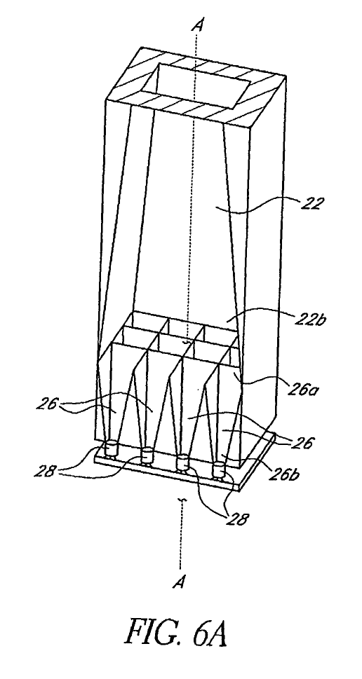

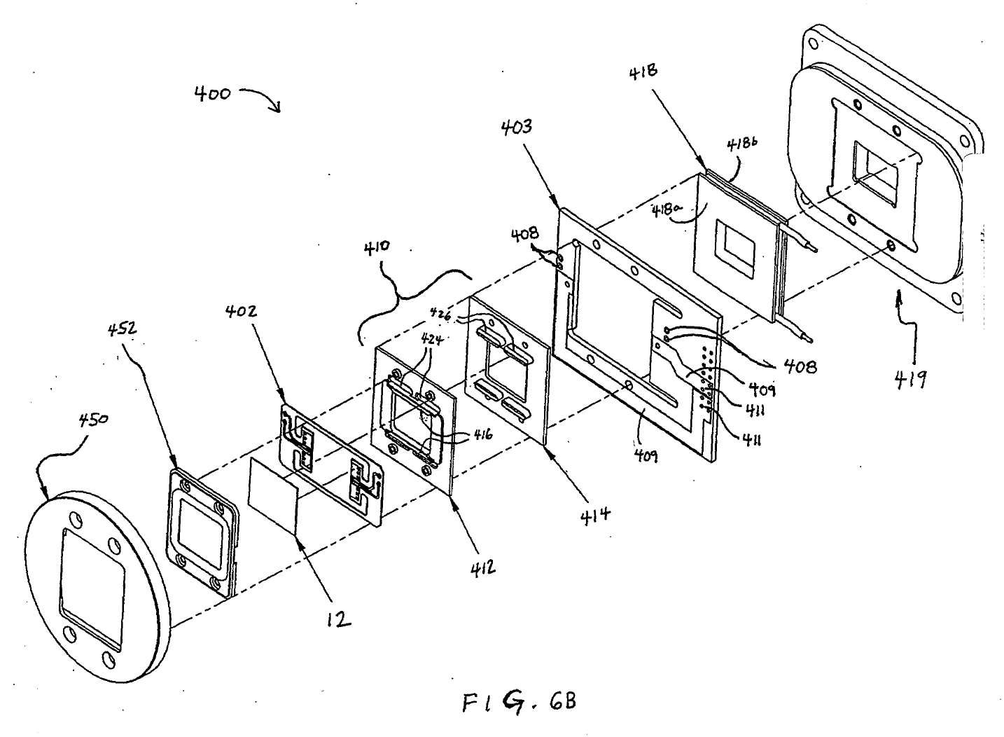

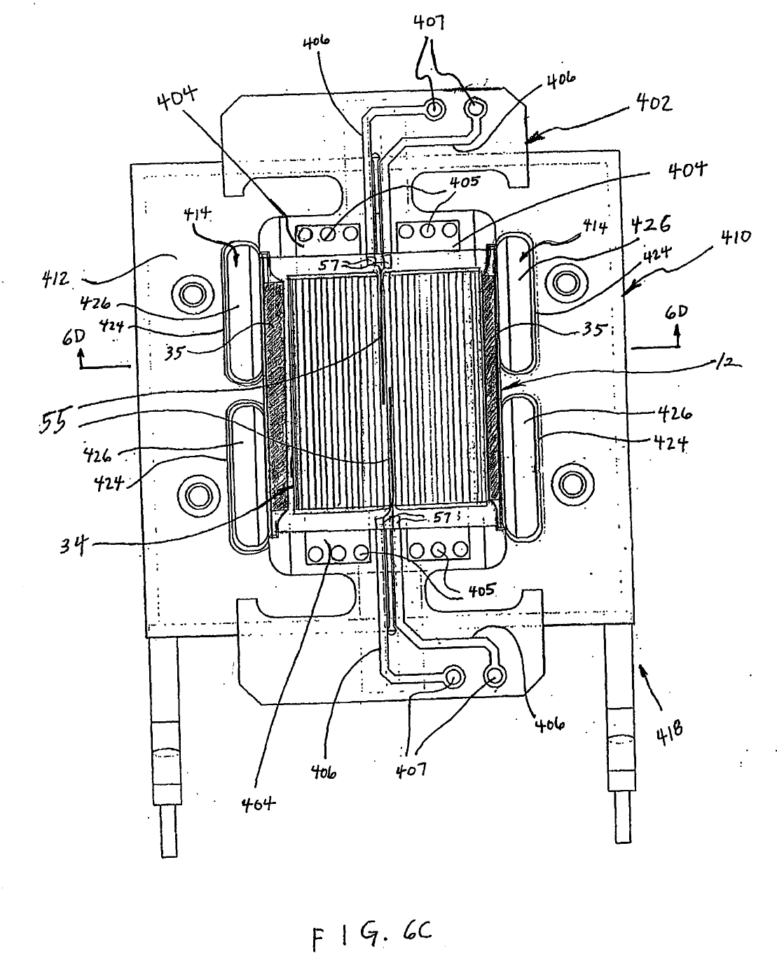

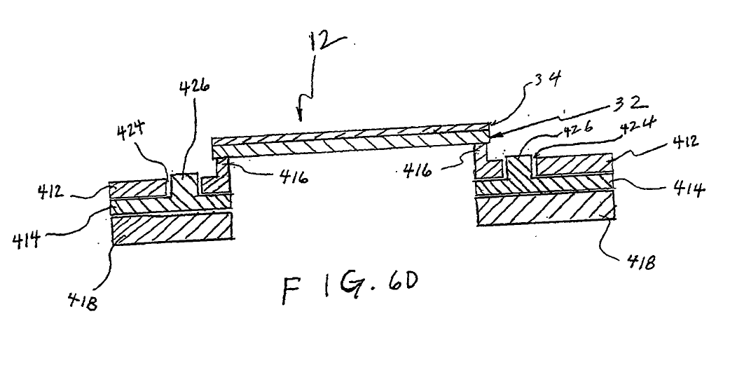

In the embodiment of FIG. 2A, the heater elements 38 are typically provided with a width of about 25 microns. The pitch distance separating centerlines of adjacent heater elements 38 may be reduced, and/or the width of the heater elements 38 may be increased, in the regions of the window assembly 12 near the point(s) of contact with the thermal diffuser 410 (see FIGS. 6B-6D and discussion below). This arrangement advantageously promotes an isothermal temperature profile at the upper surface of the main layer 32 despite thermal contact with the thermal diffuser.

The embodiment shown in FIG. 2A includes a plurality of heater elements 38 of substantially equal width which are variably spaced across the width of the main layer 32. In the embodiment of FIG. 2A, the centerlines of the heater elements 38 are spaced at a first pitch distance of about 0.0070″ at peripheral portions 34a of the heater layer 34, and at a second pitch distance of about 0.015″ at a central portion 34b of the main layer 32. The heater elements 38 closest to the center are preferably sufficiently spaced to allow the RTDs 55 to extend therebetween. In the embodiment of FIG. 2A, the main layer 32 includes peripheral regions 32a which extend about 0.053″ from the outermost heater element on each side of the heater layer 34 to the adjacent edge of the main layer 32. As shown, the bus bars 36 are preferably configured and segmented to allow space for the RTDs 55 and the RTD connection pads 57, in intermediate gaps 36a. The RTDs 55 preferably extend into the array of heater elements 38 by distance that is slightly longer than half of the length of an individual heater element 38. In alternative embodiments, the RTDs 55 may be located at the edges of the main layer 32, or at other locations as desired for a particular noninvasive system.

With continued reference to FIG. 2A, the peripheral regions of the main layer 32 may include metallized edge portions 35 for facilitating connection to the diffuser 410 (discussed below in connection with FIGS. 6B-6D). The metallized edge portions 35 may be formed by the same or similar processes used in forming the heater elements 38 and RTDs 55. In the embodiment of FIG. 2A, the edge portions 35 are typically between about 0.040″ and about 0.060″ wide by about 0.450″ and about 0.650″ long, and in one embodiment, they are about 0.050″ by about 0.550″. Other dimensions may be appropriately used so long as the window assembly 12 may be joined in thermal communication with the diffuser 410 as needed.

In the embodiment shown in FIG. 2A, the main layer 32 is about 0.690″ long by about 0.571″ wide, and the heater layer (excluding the metallized edge portions 35) is about 0.640″ long by about 0.465″ wide. The main layer 32 is about 0.010″-0.012″ thick, and is advantageously thinner than about 0.010″ where possible. Each heater element 38 is about 0.570″ long, and each peripheral region 34a is about 0.280″ wide. These dimensions are merely exemplary; of course, other dimensions may be used as desired.

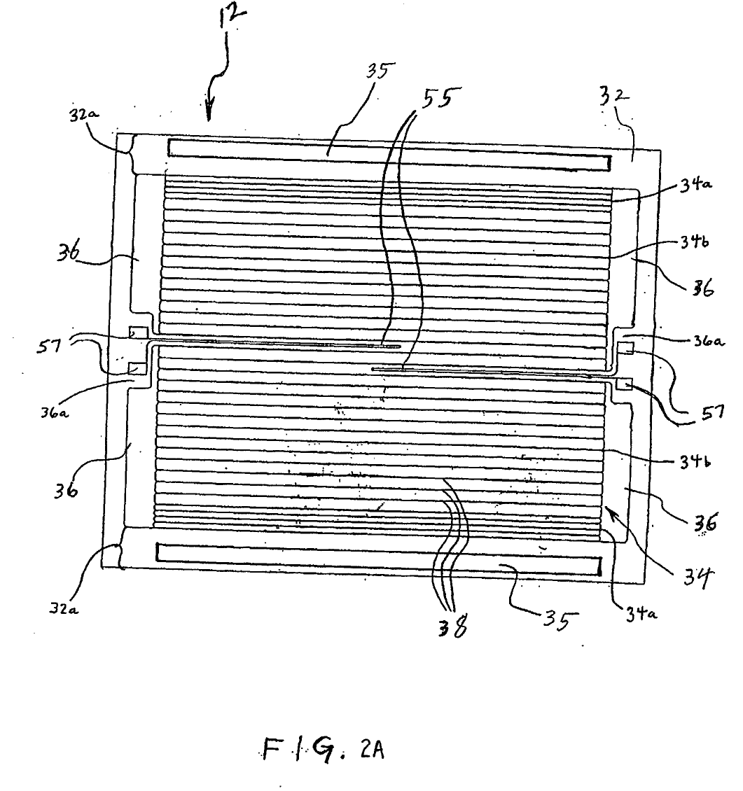

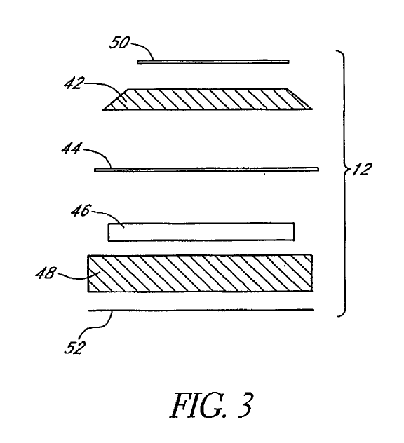

FIG. 3 depicts an exploded side view of an alternative configuration for the window assembly 12, which may be used in place of the configuration shown in FIG. 2. The window assembly 12 depicted in FIG. 3 includes near its upper surface (the surface intended for contact with the sample S) a highly infrared-transmissive, thermally conductive spreader layer 42. Underlying the spreader layer 42 is a heater layer 44. A thin electrically insulating layer (not shown), such as layer of aluminum oxide, titanium dioxide or zinc selenide, may be disposed between the heater layer 44 and the spreader layer 42. (An aluminum oxide layer also increases adhesion of the heater layer 44 to the spreader layer 42.) Adjacent to the heater layer 44 is a thermal insulating and impedance matching layer 46. Adjacent to the thermal insulating layer 46 is a thermally conductive inner layer 48. The spreader layer 42 is coated on its top surface with a thin layer of protective coating 50. The bottom surface of the inner layer 48 is coated with a thin overcoat layer 52. Preferably, the protective coating 50 and the overcoat layer 52 have antireflective properties.

The spreader layer 42 is preferably formed of a highly infrared-transmissive material having a high thermal conductivity sufficient to facilitate heat transfer from the heater layer 44 uniformly into the material sample S when it is placed against the window assembly 12. Other effective materials include, but are not limited to, CVD diamond, diamondlike carbon, gallium arsenide, germanium, and other infrared-transmissive materials having sufficiently high thermal conductivity. Preferred dimensions for the spreader layer 42 are about one inch in diameter and about 0.010 inch thick. As shown in FIG. 3, a preferred embodiment of the spreader layer 42 incorporates a beveled edge. Although not required, an approximate 45-degree bevel is preferred.

The protective layer 50 is intended to protect the top surface of the spreader layer 42 from damage. Ideally, the protective layer is highly infrared-transmissive and highly resistant to mechanical damage, such as scratching or abrasion. It is also preferred that the protective layer 50 and the overcoat layer 52 have high thermal conductivity and antireflective and/or index-matching properties. A satisfactory material for use as the protective layer 50 and the overcoat layer 52 is the multi-layer Broad Band Anti-Reflective Coating produced by Deposition Research Laboratories, Inc. of St. Charles, Mo. Diamondlike carbon coatings are also suitable.

Except as noted below, the heater layer 44 is generally similar to the heater layer 34 employed in the window assembly shown in FIG. 2. Alternatively, the heater layer 44 may comprise a doped infrared-transmissive material, such as a doped silicon layer, with regions of higher and lower resistivity. The heater layer 44 preferably has a resistance of about 2 ohms and has a preferred thickness of about 1,500 angstroms. A preferred material for forming the heater layer 44 is a gold alloy, but other acceptable materials include, but are not limited to, platinum, titanium, tungsten, copper, and nickel.

The thermal insulating layer 46 prevents the dissipation of heat from the heater element 44 while allowing the cooling system 14 to effectively cool the material sample S (see FIG. 1). This layer 46 comprises a material having thermally insulative (e.g., lower thermal conductivity than the spreader layer 42) and infrared transmissive qualities. A preferred material is a germanium-arsenic-selenium compound of the calcogenide glass family known as AMTIR-1 produced by Amorphous Materials, Inc. of Garland, Tex. The pictured embodiment has a diameter of about 0.85 inches and a preferred thickness in the range of about 0.005 to about 0.010 inches. As heat generated by the heater layer 44 passes through the spreader layer 42 into the material sample S, the thermal insulating layer 46 insulates this heat.

The inner layer 48 is formed of thermally conductive material, preferably crystalline silicon formed using a conventional floatzone crystal growth method. The purpose of the inner layer 48 is to serve as a cold-conducting mechanical base for the entire layered window assembly.

The overall optical transmission of the window assembly 12 shown in FIG. 3 is preferably at least 70%. The window assembly 12 of FIG. 3 is preferably held together and secured to the noninvasive system 10 by a holding bracket (not shown). The bracket is preferably formed of a glass-filled plastic, for example Ultem 2300, manufactured by General Electric. Ultem 2300 has low thermal conductivity which prevents heat transfer from the layered window assembly 12.

3. Cooling System

The cooling system 14 (see FIG. 1) preferably comprises a Peltier-type thermoelectric device. Thus, the application of an electrical current to the preferred cooling system 14 causes the cold surface 14a to cool and causes the opposing hot surface 14b to heat up. The cooling system 14 cools the window assembly 12 via the situation of the window assembly 12 in thermally conductive relation to the cold surface 14a of the cooling system 14. It is contemplated that the cooling system 14, the heater layer 34, or both, can be operated to induce a desired time-varying temperature in the window assembly 12 to create an oscillating thermal gradient in the sample S, in accordance with various analyte-detection methodologies discussed herein.

Preferably, the cold reservoir 16 is positioned between the cooling system 14 and the window assembly 12, and functions as a thermal conductor between the system 14 and the window assembly 12. The cold reservoir 16 is formed from a suitable thermally conductive material, preferably brass. Alternatively, the window assembly 12 can be situated in direct contact with the cold surface 14a of the cooling system 14.

In alternative embodiments, the cooling system 14 may comprise a heat exchanger through which a coolant, such as air, nitrogen or chilled water, is pumped, or a passive conduction cooler such as a heat sink. As a further alternative, a gas coolant such as nitrogen may be circulated through the interior of the noninvasive system 10 so as to contact the underside of the window assembly 12 (see FIG. 1) and conduct heat therefrom.

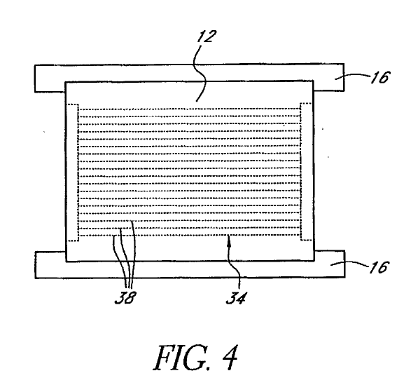

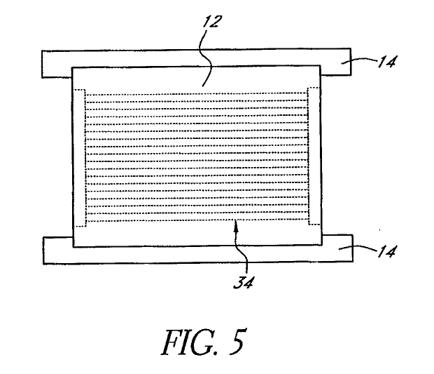

FIG. 4 is a top schematic view of a preferred arrangement of the window assembly 12 (of the types shown in FIG. 2 or 2A) and the cold reservoir 16, and FIG. 5 is a top schematic view of an alternative arrangement in which the window assembly 12 directly contacts the cooling system 14. The cold reservoir 16/cooling system 14 preferably contacts the underside of the window assembly 12 along opposing edges thereof, on either side of the heater layer 34. With thermal conductivity thus established between the window assembly 12 and the cooling system 14, the window assembly can be cooled as needed during operation of the noninvasive system 10. In order to promote a substantially uniform or isothermal temperature profile over the upper surface of the window assembly 12, the pitch distance between centerlines of adjacent heater elements 38 may be made smaller (thereby increasing the density of heater elements 38) near the region(s) of contact between the window assembly 12 and the cold reservoir 16/cooling system 14. As a supplement or alternative, the heater elements 38 themselves may be made wider near these regions of contact. As used herein, “isothermal” is a broad term and is used in its ordinary sense and refers, without limitation, to a condition in which, at a given point in time, the temperature of the window assembly 12 or other structure is substantially uniform across a surface intended for placement in thermally conductive relation to the material sample S. Thus, although the temperature of the structure or surface may fluctuate over time, at any given point in time the structure or surface may nonetheless be isothermal.

The heat sink 18 drains waste heat from the hot surface 14b of the cooling system 16 and stabilizes the operational temperature of the noninvasive system 10. The preferred heat sink 18 (see FIG. 6) comprises a hollow structure formed from brass or any other suitable material having a relatively high specific heat and high heat conductivity. The heat sink 18 has a conduction surface 18a which, when the heat sink 18 is installed in the noninvasive system 18, is in thermally conductive relation to the hot surface 14b of the cooling system 14 (see FIG. 1). A cavity 54 is formed in the heat sink 18 and preferably contains a phase-change material (not shown) to increase the capacity of the sink 18. A preferred phase change material is a hydrated salt, such as calciumchloride hexahydrate, available under the name TH29 from PCM Thermal Solutions, Inc., of Naperville, Ill. Alternatively, the cavity 54 may be omitted to create a heat sink 18 comprising a solid, unitary mass. The heat sink 18 also forms a number of fins 56 to further increase the conduction of heat from the sink 18 to surrounding air.

Alternatively, the heat sink 18 may be formed integrally with the optical mixer 20 and/or the collimator 22 as a unitary mass of rigid, heat-conductive material such as brass or aluminum. In such a heat sink, the mixer 20 and/or collimator 22 extend axially through the heat sink 18, and the heat sink defines the inner walls of the mixer 20 and/or collimator 22. These inner walls are coated and/or polished to have appropriate reflectivity and nonabsorbance in infrared wavelengths as will be further described below. Where such a unitary heat sink-mixer-collimator is employed, it is desirable to thermally insulate the detector array from the heat sink.

It should be understood that any suitable structure may be employed to heat and/or cool the material sample S, instead of or in addition to the window assembly 12/cooling system 14 disclosed above, so long a proper degree of cycled heating and/or cooling are imparted to the material sample S. In addition other forms of energy, such as but not limited to light, radiation, chemically induced heat, friction and vibration, may be employed to heat the material sample S. It will be further appreciated that heating of the sample can achieved by any suitable method, such as convection, conduction, radiation, etc.

4. Window Mounting System

FIG. 6B illustrates an exploded view of a window mounting system 400 which, in one embodiment, is employed as part of the noninvasive system 10 disclosed above. Where employed in connection with the noninvasive system 10, the window mounting system 400 supplements or, where appropriate, replaces any of the window assembly 12, cooling system 14, cold reservoir 16 and heat sink 18 shown in FIG. 1. In one embodiment, the window mounting system 400 is employed in conjunction with the window assembly 12 depicted in FIG. 2A; in alternative embodiments, the window assemblies shown in FIGS. 2 and 3 and described above may also be used in conjunction with the window mounting system 400 illustrated in FIG. 6B.

In the window mounting system 400, the window assembly 12 is physically and electrically connected (typically by soldering) to a first printed circuit board (“first PCB”) 402. The window assembly 12 is also in thermally conductive relation (typically by contact) to a thermal diffuser 410. The window assembly may also be fixed to the diffuser 410 by soldering.

The thermal diffuser 410 generally comprises a heat spreader layer 412 which, as mentioned, preferably contacts the window assembly 12, and a conductive layer 414 which is typically soldered to the heat spreader layer 412. The conductive layer 414 may then be placed in direct contact with a cold side 418a of a thermoelectric cooler (TEC) 418 or other cooling device. The TEC 418, which in one embodiment comprises a 25 W TEC manufactured by MELCOR, is in electrical communication with a second PCB 403, which includes TEC power leads 409 and TEC power terminals 411 for connection of the TEC 418 to an appropriate power source (not shown). The second PCB 403 also includes contacts 408 for connection with RTD terminals 407 (see FIG. 6C) of the first PCB 402. A heat sink 419, which may take the form of the illustrated water jacket, the heat sink 18 shown in FIG. 6, any other heat sink structures mentioned herein, or any other appropriate device, is in thermal communication with a hot side 418b of the TEC 418 (or other cooling device), in order to remove any excess heat created by the TEC 418.

FIG. 6C illustrates a plan view of the interconnection of the window assembly 12, the first PCB 402, the diffuser 410 and the thermoelectric cooler 418. The first PCB includes RTD bonding leads 406 and heater bonding pads 404 which permit attachment of the RTDs 55 and bus bars 36, respectively, of the window assembly 12 to the first PCB 402 via soldering or other conventional techniques. Electrical communication is thus established between the heater elements 38 of the heater layer 34, and heater terminals 405 formed in the heater bonding pads 404. Similarly, electrical communication is established between the RTDs 55 and RTD terminals 407 formed at the ends of the RTD bonding leads 406. Electrical connections can be established with the heater elements 38 and the RTDs 55 via simple connection to the terminals 405, 407 of the first PCB 402.

With further reference to FIGS. 2A and 6B-6C, the heat spreader layer 412 of the thermal diffuser 410 contacts the underside of the main layer 32 of the window assembly 12 via a pair of rails 416. The rails 416 may contact the main layer 32 at the metallized edge portions 35, or at any other appropriate location. The physical and thermal connection between the rails 416 and the window main layer 32 may be achieved by soldering, as indicated above. Alternatively, the connection may be achieved by an adhesive such as epoxy, or any other appropriate method. The material chosen for the window main layer 32 is preferably sufficiently thermally conductive that heat may be quickly removed from the main layer 32 through the rails 416, the diffuser 410, and the TEC 128.

FIG. 6D shows a cross-sectional view of the assembly of FIG. 6C through line 22-22. As can be seen in FIG. 6D, the window assembly 12 contacts the rails 416 of the heat spreader layer 412. The conductive layer 414 underlies the spreader layer 412 and may comprise protrusions 426 configured to extend through openings 424 formed in the spreader layer 412. The openings 424 and protrusions 426 are sized to leave sufficient expansion space therebetween, to allow expansion and contraction of the conductive layer 414 without interference with, or causing deformation of, the window assembly 12 or the heat spreader layer 412. Moreover, the protrusions 426 and openings 424 coact to prevent displacement of the spreader layer 412 with respect to the conductive layer 414 as the conductive layer 414 expands and contracts.

The thermal diffuser 410 provides a thermal impedance between the TEC 418 and the window assembly 12, which impedance is selected to drain heat from the window assembly at a rate proportional to the power output of the heater layer 34. In this way, the temperature of the main layer 32 can be rapidly cycled between a “hot” and a “cold” temperatures, thereby allowing a time-varying thermal gradient to be induced in a sample S placed against the window assembly 12.

The heat spreader layer 412 is preferably made of a material which has substantially the same coefficient of thermal expansion as the material used to form the window assembly main layer 32, within the expected operating temperature range. Preferably, both the material used to form the main layer 32 and the material used to form the heat spreader layer 412 have substantially the same, extremely low, coefficient of thermal expansion. For this reason, CVD diamond is preferred for the main layer 32 (as mentioned above); with a CVD diamond main layer 32 the preferred material for the heat spreader layer 412 is Invar. Invar advantageously has an extremely low coefficient of thermal expansion and a relatively high thermal conductivity. Because Invar is a metal, the main layer 32 and the heat spreader layer 412 can be thermally bonded to one another with little difficulty. Alternatively, other materials may be used for the heat spreader layer 412; for example, any of a number of glass and ceramic materials with low coefficients of thermal expansion may be employed.

The conductive layer 414 of the thermal diffuser 410 is typically a highly thermally conductive material such as copper (or, alternatively, other metals or non-metals exhibiting comparable thermal conductivities). The conductive layer 414 is typically soldered or otherwise bonded to the underside of the heat spreader layer 412.

In the illustrated embodiment, the heat spreader layer 412 may be constructed according to the following dimensions, which are to be understood as exemplary; accordingly the dimensions may be varied as desired. The heat spreader layer 412 has an overall length and width of about 1.170″, with a central opening of about 0.590″ long by 0.470″ wide. Generally, the heat spreader layer 412 is about 0.030″ thick; however, the rails 416 extend a further 0.045″ above the basic thickness of the heat spreader layer 412. Each rail 416 has an overall length of about 0.710″; over the central 0.525″ of this length each rail 416 is about 0.053″ wide. On either side of the central width each rail 416 tapers, at a radius of about 0.6″, down to a width of about 0.023″. Each opening 424 is about 0.360″ long by about 0.085″ wide, with corners rounded at a radius of about 0.033″.

In the illustrated embodiment, conductive layer 414 may be constructed according to the following dimensions, which are to be understood as exemplary; accordingly the dimensions may be varied as desired. The conductive layer 414 has an overall length and width of about 1.170″, with a central opening of about 0.590″ long by 0.470″ wide. Generally, the conductive layer 412 is about 0.035″ thick; however, the protrusions 426 extend a further 0.075″-0.085″ above the basic thickness of the conductive layer 414. Each protrusion 426 is about 0.343″ long by about 0.076″ wide, with corners rounded at a radius of about 0.035″.

As shown in FIG. 6B, first and second clamping plates 450 and 452 may be used to clamp the portions of the window mounting system 400 to one another. For example, the second clamping plate 452 is configured to clamp the window assembly 12 and the first PCB 402 to the diffuser 410 with screws or other fasteners extending through the openings shown in the second clamping plate 452, the heat spreader layer 412 and the conductive layer 414. Similarly, the first clamping plate 450 is configured overlie the second clamping plate 452 and clamp the rest of the window mounting system 400 to the heat sink 419, thus sandwiching the second clamping plate 452, the window assembly 12, the first PCB 402, the diffuser 410, the second PCB 403, and the TEC 418 therebetween. The first clamping plate 450 prevents undesired contact between the sample S and any portion of the window mounting system 400, other than the window assembly 12 itself. Other mounting plates and mechanisms may also be used as desired.

5. Optics

As shown in FIG. 1, the optical mixer 20 comprises a light pipe with an inner surface coating which is highly reflective and minimally absorptive in infrared wavelengths, preferably a polished gold coating, although other suitable coatings may be used where other wavelengths of electromagnetic radiation are employed. The pipe itself may be fabricated from a another rigid material such as aluminum or stainless steel, as long as the inner surfaces are coated or otherwise treated to be highly reflective. Preferably, the optical mixer 20 has a rectangular cross-section (as taken orthogonal to the longitudinal axis A-A of the mixer 20 and the collimator 22), although other cross-sectional shapes, such as other polygonal shapes or circular or elliptical shapes, may be employed in alternative embodiments. The inner walls of the optical mixer 20 are substantially parallel to the longitudinal axis A-A of the mixer 20 and the collimator 22. The highly reflective and substantially parallel inner walls of the mixer 20 maximize the number of times the infrared energy E will be reflected between the walls of the mixer 20, thoroughly mixing the infrared energy E as it propagates through the mixer 20. In a presently preferred embodiment, the mixer 20 is about 1.2 inches to 2.4 inches in length and its cross-section is a rectangle of about 0.4 inches by about 0.6 inches. Of course, other dimensions may be employed in constructing the mixer 20. In particular it is be advantageous to miniaturize the mixer or otherwise make it as small as possible

Still referring to FIG. 1, the collimator 22 comprises a tube with an inner surface coating which is highly reflective and minimally absorptive in infrared wavelengths, preferably a polished gold coating. The tube itself may be fabricated from a another rigid material such as aluminum, nickel or stainless steel, as long as the inner surfaces are coated or otherwise treated to be highly reflective. Preferably, the collimator 22 has a rectangular cross-section, although other cross-sectional shapes, such as other polygonal shapes or circular, parabolic or elliptical shapes, may be employed in alternative embodiments. The inner walls of the collimator 22 diverge as they extend away from the mixer 20. Preferably, the inner walls of the collimator 22 are substantially straight and form an angle of about 7 degrees with respect to the longitudinal axis A-A. The collimator 22 aligns the infrared energy E to propagate in a direction that is generally parallel to the longitudinal axis A-A of the mixer 20 and the collimator 22, so that the infrared energy E will strike the surface of the filters 24 at an angle as close to 90 degrees as possible.

In a presently preferred embodiment, the collimator is about 7.5 inches in length. At its narrow end 22a, the cross-section of the collimator 22 is a rectangle of about 0.4 inches by 0.6 inches. At its wide end 22b, the collimator 22 has a rectangular cross-section of about 1.8 inches by 2.6 inches. Preferably, the collimator 22 aligns the infrared energy E to an angle of incidence (with respect to the longitudinal axis A-A) of about 0-15 degrees before the energy E impinges upon the filters 24. Of course, other dimensions or incidence angles may be employed in constructing and operating the collimator 22.

With further reference to FIGS. 1 and 6A, each concentrator 26 comprises a tapered surface oriented such that its wide end 26a is adapted to receive the infrared energy exiting the corresponding filter 24, and such that its narrow end 26b is adjacent to the corresponding detector 28. The inward-facing surfaces of the concentrators 26 have an inner surface coating which is highly reflective and minimally absorptive in infrared wavelengths, preferably a polished gold coating. The concentrators 26 themselves may be fabricated from a another rigid material such as aluminum, nickel or stainless steel, so long as their inner surfaces are coated or otherwise treated to be highly reflective.

Preferably, the concentrators 26 have a rectangular cross-section (as taken orthogonal to the longitudinal axis A-A), although other cross-sectional shapes, such as other polygonal shapes or circular, parabolic or elliptical shapes, may be employed in alternative embodiments. The inner walls of the concentrators converge as they extend toward the narrow end 26b. Preferably, the inner walls of the collimators 26 are substantially straight and form an angle of about 8 degrees with respect to the longitudinal axis A-A. Such a configuration is adapted to concentrate infrared energy as it passes through the concentrators 26 from the wide end 26a to the narrow end 26b, before reaching the detectors 28.

In a presently preferred embodiment, each concentrator 26 is about 1.5 inches in length. At the wide end 26a, the cross-section of each concentrator 26 is a rectangle of about 0.6 inches by 0.57 inches. At the narrow end 26b, each concentrator 26 has a rectangular cross-section of about 0.177 inches by 0.177 inches. Of course, other dimensions or incidence angles may be employed in constructing the concentrators 26.

6. Filters

The filters 24 preferably comprise standard interference-type infrared filters, widely available from manufacturers such as Optical Coating Laboratory, Inc. (“OCLI”) of Santa Rosa, Calif. In the embodiment illustrated in FIG. 1, a 3×4 array of filters 24 is positioned above a 3×4 array of detectors 28 and concentrators 26. As employed in this embodiment, the filters 24 are arranged in four groups of three filters having the same wavelength sensitivity. These four groups have bandpass center wavelengths of 7.15 μm±0.03 μm, 8.40 μm±0.03 μm, 9.48 μm±0.04 μm, and 11.10 μm±0.04 μm, respectively, which correspond to wavelengths around which water and glucose absorb electromagnetic radiation. Typical bandwidths for these filters range from 0.20 μm to 0.50 μm.

In an alternative embodiment, the array of wavelength-specific filters 24 may be replaced with a single Fabry-Perot interferometer, which can provide wavelength sensitivity which varies as a sample of infrared energy is taken from the material sample S. Thus, this embodiment permits the use of only one detector 28, the output signal of which varies in wavelength specificity over time. The output signal can be de-multiplexed based on the wavelength sensitivities induced by the Fabry-Perot interferometer, to provide a multiple-wavelength profile of the infrared energy emitted by the material sample S. In this embodiment, the optical mixer 20 may be omitted, as only one detector 28 need be employed.

In still other embodiments, the array of filters 24 may comprise a filter wheel that rotates different filters with varying wavelength sensitivities over a single detector 24. Alternatively, an electronically tunable infrared filter may be employed in a manner similar to the Fabry-Perot interferometer discussed above, to provide wavelength sensitivity which varies during the detection process. In either of these embodiments, the optical mixer 20 may be omitted, as only one detector 28 need be employed.

7. Detectors

The detectors 28 may comprise any detector type suitable for sensing infrared energy, preferably in the mid-infrared wavelengths. For example, the detectors 28 may comprise mercury-cadmium-telluride (MCT) detectors. A detector such as a Fermionics (Simi Valley, Calif.) model PV-9.1 with a PVA481-1 pre-amplifier is acceptable. Similar units from other manufacturers such as Graseby (Tampa, Fla.) can be substituted. Other suitable components for use as the detectors 28 include pyroelectric detectors, thermopiles, bolometers, silicon microbolometers and lead-salt focal plane arrays.

8. Control System

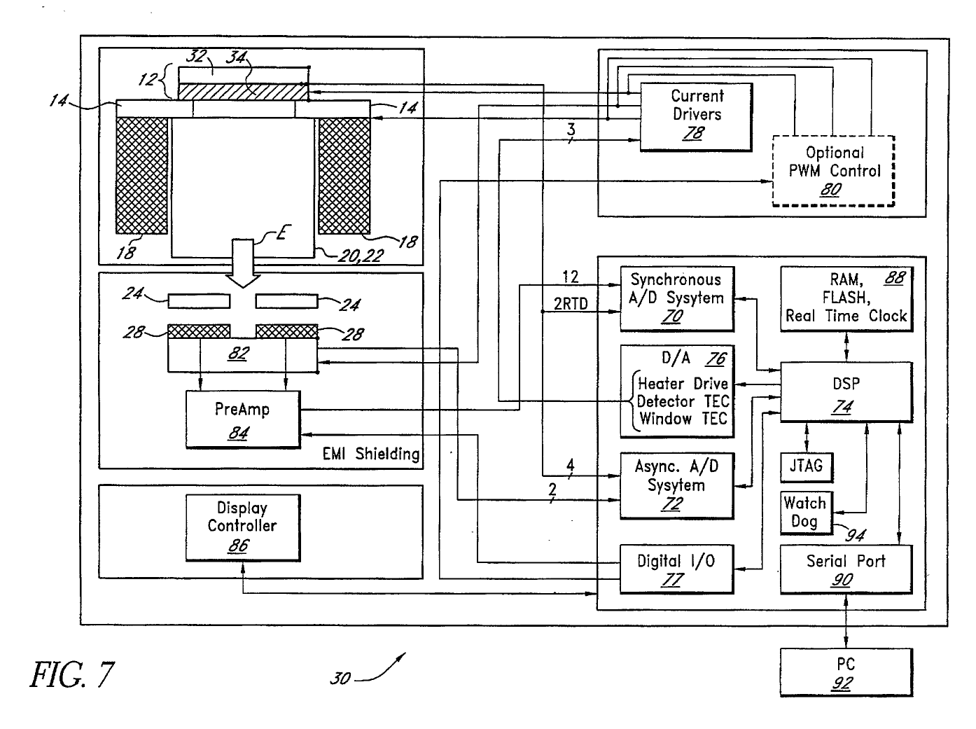

FIG. 7 depicts the control system 30 in greater detail, as well as the interconnections between the control system and other relevant portions of the noninvasive system. The control system includes a temperature control subsystem and a data acquisition subsystem.

In the temperature control subsystem, temperature sensors (such as RTDs and/or thermistors) located in the window assembly 12 provide a window temperature signal to a synchronous analog-to-digital conversion system 70 and an asynchronous analog-to-digital conversion system 72. The A/D systems 70, 72 in turn provide a digital window temperature signal to a digital signal processor (DSP) 74. The processor 74 executes a window temperature control algorithm and determines appropriate control inputs for the heater layer 34 of the window assembly 12 and/or for the cooling system 14, based on the information contained in the window temperature signal. The processor 74 outputs one or more digital control signals to a digital-to-analog conversion system 76 which in turn provides one or more analog control signals to current drivers 78. In response to the control signal(s), the current drivers 78 regulate the power supplied to the heater layer 34 and/or to the cooling system 14. In one embodiment, the processor 74 provides a control signal through a digital I/O device 77 to a pulse-width modulator (PWM) control 80, which provides a signal that controls the operation of the current drivers 78. Alternatively, a low-pass filter (not shown) at the output of the PWM provides for continuous operation of the current drivers 78.

In another embodiment, temperature sensors may be located at the cooling system 14 and appropriately connected to the A/D system(s) and processor to provide closed-loop control of the cooling system as well.

In yet another embodiment, a detector cooling system 82 is located in thermally conductive relation to one or more of the detectors 28. The detector cooling system 82 may comprise any of the devices disclosed above as comprising the cooling system 14, and preferably comprises a Peltier-type thermoelectric device. The temperature control subsystem may also include temperature sensors, such as RTDs and/or thermistors, located in or adjacent to the detector cooling system 82, and electrical connections between these sensors and the asynchronous A/D system 72. The temperature sensors of the detector cooling system 82 provide detector temperature signals to the processor 74. In one embodiment, the detector cooling system 82 operates independently of the window temperature control system, and the detector cooling system temperature signals are sampled using the asynchronous A/D system 72. In accordance with the temperature control algorithm, the processor 74 determines appropriate control inputs for the detector cooling system 82, based on the information contained in the detector temperature signal. The processor 74 outputs digital control signals to the D/A system 76 which in turn provides analog control signals to the current drivers 78. In response to the control signals, the current drivers 78 regulate the power supplied to the detector cooling system 14. In one embodiment, the processor 74 also provides a control signal through the digital I/O device 77 and the PWM control 80, to control the operation of the detector cooling system 82 by the current drivers 78. Alternatively, a low-pass filter (not shown) at the output of the PWM provides for continuous operation of the current drivers 78.

In the data acquisition subsystem, the detectors 28 respond to the infrared energy E incident thereon by passing one or more analog detector signals to a preamp 84. The preamp 84 amplifies the detector signals and passes them to the synchronous A/D system 70, which converts the detector signals to digital form and passes them to the processor 74. The processor 74 determines the concentrations of the analyte(s) of interest, based on the detector signals and a concentration-analysis algorithm and/or phase/concentration regression model stored in a memory module 88. The concentration-analysis algorithm and/or phase/concentration regression model may be developed according to any of the analysis methodologies discussed herein. The processor may communicate the concentration results and/or other information to a display controller 86, which operates a display (not shown), such as an LCD display, to present the information to the user.

A watchdog timer 94 may be employed to ensure that the processor 74 is operating correctly. If the watchdog timer 94 does not receive a signal from the processor 74 within a specified time, the watchdog timer 94 resets the processor 74. The control system may also include a JTAG interface 96 to enable testing of the noninvasive system 10.

In one embodiment, the synchronous A/D system 70 comprises a 20-bit, 14 channel system, and the asynchronous A/D system 72 comprises a 16-bit, 16 channel system. The preamp may comprise a 12-channel preamp corresponding to an array of 12 detectors 28.

The control system may also include a serial port 90 or other conventional data port to permit connection to a personal computer 92. The personal computer can be employed to update the algorithm(s) and/or phase/concentration regression model(s) stored in the memory module 88, or to download a compilation of analyte-concentration data from the noninvasive system. A real-time clock or other timing device may be accessible by the processor 74 to make any time-dependent calculations which may be desirable to a user.

9. Analysis Methodology

The detector(s) 28 of the noninvasive system 10 are used to detect the infrared energy emitted by the material sample S in various desired wavelengths. At each measured wavelength, the material sample S emits infrared energy at an intensity which varies over time. The time-varying intensities arise largely in response to the use of the window assembly 12 (including its heater layer 34) and the cooling system 14 to induce a thermal gradient in the material sample S. As used herein, “thermal gradient” is a broad term and is used in its ordinary sense and refers, without limitation, to a difference in temperature and/or thermal energy between different locations, such as different depths, of a material sample, which can be induced by any suitable method of increasing or decreasing the temperature and/or thermal energy in one or more locations of the sample. As will be discussed in detail below, the concentration of an analyte of interest (such as glucose) in the material sample S can be determined with a device such as the noninvasive system 10, by comparing the time-varying intensity profiles of the various measured wavelengths.

Analysis methodologies are discussed herein within the context of detecting the concentration of glucose within a material sample, such as a tissue sample, which includes a large proportion of water. However, it will evident that these methodologies are not limited to this context and may be applied to the detection of a wide variety of analytes within a wide variety of sample types. It should also be understood that other suitable analysis methodologies and suitable variations of the disclosed methodologies may be employed in operating an analyte detection system, such as the noninvasive system 10.

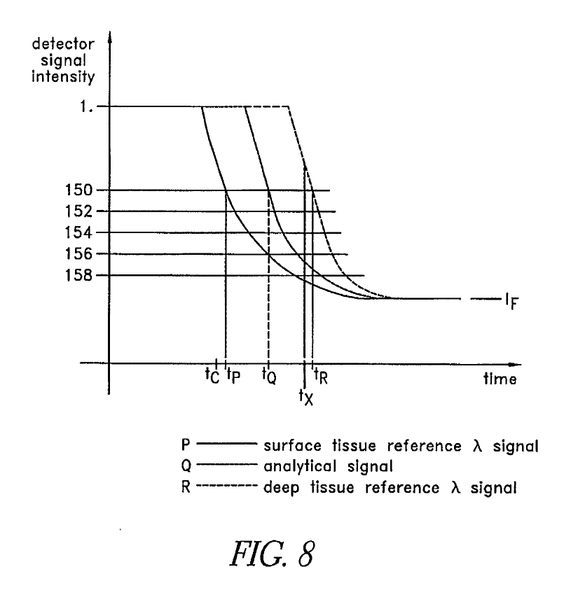

As shown in FIG. 8, a first reference signal P may be measured at a first reference wavelength. The first reference signal P is measured at a wavelength where water strongly absorbs (e.g., 2.9 μm or 6.1 μm). Because water strongly absorbs radiation at these wavelengths, the detector signal intensity is reduced at those wavelengths. Moreover, at these wavelengths water absorbs the photon emissions emanating from deep inside the sample. The net effect is that a signal emitted at these wavelengths from deep inside the sample is not easily detected. The first reference signal P is thus a good indicator of thermal-gradient effects near the sample surface and may be known as a surface reference signal. This signal may be calibrated and normalized, in the absence of heating or cooling applied to the sample, to a baseline value of 1. For greater accuracy, more than one first reference wavelength may be measured. For example, both 2.9 μm and 6.1 μm may be chosen as first reference wavelengths.

As further shown in FIG. 8, a second reference signal R may also be measured. The second signal R may be measured at a wavelength where water has very low absorbance (e.g., 3.6 μm or 4.2 μm). This second reference signal R thus provides the analyst with information concerning the deeper regions of the sample, whereas the first signal P provides information concerning the sample surface. This signal may also be calibrated and normalized, in the absence of heating or cooling applied to the sample, to a baseline value of 1. As with the first (surface) reference signal P, greater accuracy may be obtained by using more than one second (deep) reference signal R.

In order to determine analyte concentration, a third (analytical) signal Q is also measured. This signal is measured at an IR absorbance peak of the selected analyte. The IR absorbance peaks for glucose are in the range of about 6.5 μm to 11.0 μm. This detector signal may also be calibrated and normalized, in the absence of heating or cooling applied to the material sample S, to a baseline value of 1. As with the reference signals P, R, the analytical signal Q may be measured at more than one absorbance peak.

Optionally, or additionally, reference signals may be measured at wavelengths that bracket the analyte absorbance peak. These signals may be advantageously monitored at reference wavelengths which do not overlap the analyte absorbance peaks. Further, it is advantageous to measure reference wavelengths at absorbance peaks which do not overlap the absorbance peaks of other possible constituents contained in the sample.

10. Basic Thermal Gradient

As further shown in FIG. 8, the signal intensities P, Q, R are shown initially at the normalized baseline signal intensity of 1. This of course reflects the baseline radiative behavior of a test sample in the absence of applied heating or cooling. At a time tC, the surface of the sample is subjected to a temperature event which induces a thermal gradient in the sample. The gradient can be induced by heating or cooling the sample surface. The example shown in FIG. 8 uses cooling, for example, using a 10° C. cooling event. In response to the cooling event, the intensities of the detector signals P, Q, R decrease over time.

Since the cooling of the sample is neither uniform nor instantaneous, the surface cools before the deeper regions of the sample cool. As each of the signals P, Q, R drop in intensity, a pattern emerges. Signal intensity declines as expected, but as the signals P, Q, R reach a given amplitude value (or series of amplitude values: 150, 152, 154, 156, 158), certain temporal effects are noted. After the cooling event is induced at tC, the first (surface) reference signal P declines in amplitude most rapidly, reaching a checkpoint 150 first, at time tP. This is due to the fact that the first reference signal P mirrors the sample's radiative characteristics near the surface of the sample. Since the sample surface cools before the underlying regions, the surface (first) reference signal P drops in intensity first.

Simultaneously, the second reference signal R is monitored. Since the second reference signal R corresponds to the radiation characteristics of deeper regions of the sample, which do not cool as rapidly as the surface (due to the time needed for the surface cooling to propagate into the deeper regions of the sample), the intensity of signal R does not decline until slightly later. Consequently, the signal R does not reach the magnitude 150 until some later time tR. In other words, there exists a time delay between the time tP at which the amplitude of the first reference signal P reaches the checkpoint 150 and the time tR at which the second reference signal R reaches the same checkpoint 150. This time delay can be expressed as a phase difference Φ(λ). Additionally, a phase difference may be measured between the analytical signal Q and either or both reference signals P, R.

As the concentration of analyte increases, the amount of absorbance at the analytical wavelength increases. This reduces the intensity of the analytical signal Q in a concentration-dependent way. Consequently, the analytical signal Q reaches intensity 150 at some intermediate time tQ. The higher the concentration of analyte, the more the analytical signal Q shifts to the left in FIG. 8. As a result, with increasing analyte concentration, the phase difference Φ(λ) decreases relative to the first (surface) reference signal P and increases relative to the second (deep tissue) reference signal R. The phase difference(s) Φ(λ) are directly related to analyte concentration and can be used to make accurate determinations of analyte concentration.

The phase difference Φ(λ) between the first (surface) reference signal P and the analytical signal Q is represented by the equation:

Φ(λ)=|tₚ−t_{Q}|

The magnitude of this phase difference decreases with increasing analyte concentration.

The phase difference Φ(λ) between the second (deep tissue) reference signal R and the analytical signal Q signal is represented by the equation:

Φ(λ)=|t_{Q}−t_{R}|

The magnitude of this phase difference increases with increasing analyte concentration.

Accuracy may be enhanced by choosing several checkpoints, for example, 150, 152, 154, 156, and 158 and averaging the phase differences observed at each checkpoint. The accuracy of this method may be further enhanced by integrating the phase difference(s) continuously over the entire test period. Because in this example only a single temperature event (here, a cooling event) has been induced, the sample reaches a new lower equilibrium temperature and the signals stabilize at a new constant level IF. Of course, the method works equally well with thermal gradients induced by heating or by the application or introduction of other forms of energy, such as but not limited to light, radiation, chemically induced heat, friction and vibration.

This methodology is not limited to the determination of phase difference. At any given time (for example, at a time tX) the amplitude of the analytical signal Q may be compared to the amplitude of either or both of the reference signals P, R. The difference in amplitude may be observed and processed to determine analyte concentration.

This method, the variants disclosed herein, and the apparatus disclosed as suitable for application of the method(s), are not limited to the detection of in-vivo glucose concentration. The method and disclosed variants and apparatus may be used on human, animal, or even plant subjects, or on organic or inorganic compositions in a non-medical setting. The method may be used to take measurements of in-vivo or in-vitro samples of virtually any kind. The method is useful for measuring the concentration of a wide range of additional chemical analytes, including but not limited to, glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, hormones, as well as other chemical compounds. To detect a given analyte, one needs only to select appropriate analytical and reference wavelengths.

The method is adaptable and may be used to determine chemical concentrations in samples of body fluids (e.g., blood, urine or saliva) once they have been extracted from a patient. In fact, the method may be used for the measurement of in-vitro samples of virtually any kind.

11. Modulated Thermal Gradient

In some embodiments of the methodology described above, a periodically modulated thermal gradient can be employed to make accurate determinations of analyte concentration.

As previously shown in FIG. 8, once a thermal gradient is induced in the sample, the reference and analytical signals P, Q, R fall out of phase with respect to each other. This phase difference Φ(λ) is present whether the thermal gradient is induced through heating or cooling. By alternatively subjecting the test sample to cyclic pattern of heating, cooling, or alternately heating and cooling, an oscillating thermal gradient may be induced in a sample for an extended period of time.

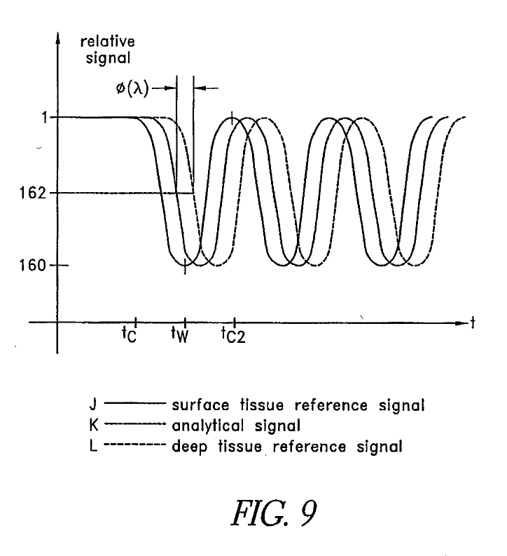

An oscillating thermal gradient is illustrated using a sinusoidally modulated gradient. FIG. 9 depicts detector signals emanating from a test sample. As with the methodology shown in FIG. 8, one or more reference signals J, L are measured. One or more analytical signals K are also monitored. These signals may be calibrated and normalized, in the absence of heating or cooling applied to the sample, to a baseline value of 1. FIG. 9 shows the signals after normalization. At some time tC, a temperature event (e.g., cooling) is induced at the sample surface. This causes a decline in the detector signal. As shown in FIG. 8, the signals (P, Q, R) decline until the thermal gradient disappears and a new equilibrium detector signal IF is reached. In the method shown in FIG. 9, as the gradient begins to disappear at a signal intensity 160, a heating event, at a time tW, is induced in the sample surface. As a result the detector output signals J, K, L will rise as the sample temperature rises. At some later time tC2, another cooling event is induced, causing the temperature and detector signals to decline. This cycle of cooling and heating may be repeated over a time interval of arbitrary length. Moreover, if the cooling and heating events are timed properly, a periodically modulated thermal gradient may be induced in the test sample.

As previously explained in the discussions relating to FIG. 8, the phase difference Φ(λ) may be measured and used to determine analyte concentration.

FIG. 9 shows that the first (surface) reference signal J declines and rises in intensity first. The second (deep tissue) reference signal L declines and rises in a time-delayed manner relative to the first reference signal J. The analytical signal K exhibits a time/phase delay dependent on the analyte concentration. With increasing concentration, the analytical signal K shifts to the left in FIG. 9. As with FIG. 8, the phase difference Φ(λ) may be measured. For example, a phase difference Φ(λ) between the second reference signal L and the analytical signal K, may be measured at a set amplitude 162 as shown in FIG. 9. Again, the magnitude of the phase signal reflects the analyte concentration of the sample.

The phase-difference information compiled by any of the methodologies disclosed herein can correlated by the control system 30 (see FIG. 1) with previously determined phase-difference information to determine the analyte concentration in the sample. This correlation could involve comparison of the phase-difference information received from analysis of the sample, with a data set containing the phase-difference profiles observed from analysis of wide variety of standards of known analyte concentration. In one embodiment, a phase/concentration curve or regression model is established by applying regression techniques to a set of phase-difference data observed in standards of known analyte concentration. This curve is used to estimate the analyte concentration in a sample based on the phase-difference information received from the sample.

Advantageously, the phase difference Φ(λ) may be measured continuously throughout the test period. The phase-difference measurements may be integrated over the entire test period for an extremely accurate measure of phase difference Φ(λ). Accuracy may also be improved by using more than one reference signal and/or more than one analytical signal.

As an alternative or as a supplement to measuring phase difference(s), differences in amplitude between the analytical and reference signal(s) may be measured and employed to determine analyte concentration. Additional details relating to this technique and not necessary to repeat here may be found in the Assignee's U.S. patent application Ser. No. 09/538,164, incorporated by reference below.

Additionally, these methods may be advantageously employed to simultaneously measure the concentration of one or more analytes. By choosing reference and analyte wavelengths that do not overlap, phase differences can be simultaneously measured and processed to determine analyte concentrations. Although FIG. 9 illustrates the method used in conjunction with a sinusoidally modulated thermal gradient, the principle applies to thermal gradients conforming to any periodic function. In more complex cases, analysis using signal processing with Fourier transforms or other techniques allows accurate determinations of phase difference Φ(λ) and analyte concentration.

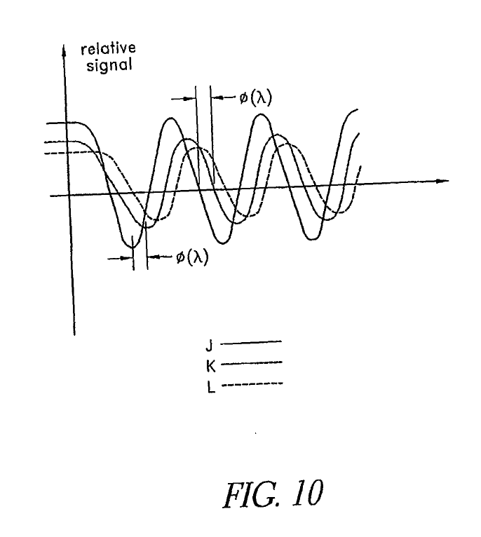

As shown in FIG. 10, the magnitude of the phase differences may be determined by measuring the time intervals between the amplitude peaks (or troughs) of the reference signals J, L and the analytical signal K. Alternatively, the time intervals between the “zero crossings” (the point at which the signal amplitude changes from positive to negative, or negative to positive) may be used to determine the phase difference between the analytical signal K and the reference signals J, L. This information is subsequently processed and a determination of analyte concentration may then be made. This particular method has the advantage of not requiring normalized signals.

As a further alternative, two or more driving frequencies may be employed to determine analyte concentrations at selected depths within the sample. A slow (e.g., 1 Hz) driving frequency creates a thermal gradient which penetrates deeper into the sample than the gradient created by a fast (e.g., 3 Hz) driving frequency. This is because the individual heating and/or cooling events are longer in duration where the driving frequency is lower. Thus, the use of a slow driving frequency provides analyte-concentration information from a deeper “slice” of the sample than does the use of a fast driving frequency.

It has been found that when analyzing a sample of human skin, a temperature event of 10° C. creates a thermal gradient which penetrates to a depth of about 150 μm, after about 500 ms of exposure. Consequently, a cooling/heating cycle or driving frequency of 1 Hz provides information to a depth of about 150 μm. It has also been determined that exposure to a temperature event of 10° C. for about 167 ms creates a thermal gradient that penetrates to a depth of about 50 μm. Therefore, a cooling/heating cycle of 3 Hz provides information to a depth of about 50 μm. By subtracting the detector signal information measured at a 3 Hz driving frequency from the detector signal information measured at, a 1 Hz driving frequency, one can determine the analyte concentration(s) in the region of skin between 50 and 150 μm. Of course, a similar approach can be used to determine analyte concentrations at any desired depth range within any suitable type of sample.

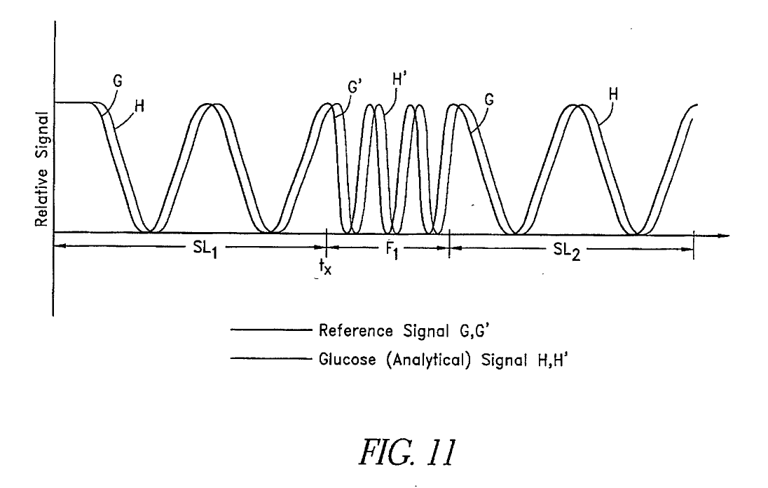

As shown in FIG. 11, alternating deep and shallow thermal gradients may be induced by alternating slow and fast driving frequencies. As with the methods described above, this variation also involves the detection and measurement of phase differences Φ(λ) between reference signals G, G′ and analytical signals H, H′. Phase differences are measured at both fast (e.g., 3 Hz) and slow (e.g., 1 Hz) driving frequencies. The slow driving frequency may continue for an arbitrarily chosen number of cycles (in region SL1), for example, two full cycles. Then the fast driving frequency is employed for a selected duration, in region F1. The phase difference data is compiled in the same manner as disclosed above. In addition, the fast frequency (shallow sample) phase difference data may be subtracted from the slow frequency (deep sample) data to provide an accurate determination of analyte concentration in the region of the sample between the gradient penetration depth associated with the fast driving frequency and that associated with the slow driving frequency.

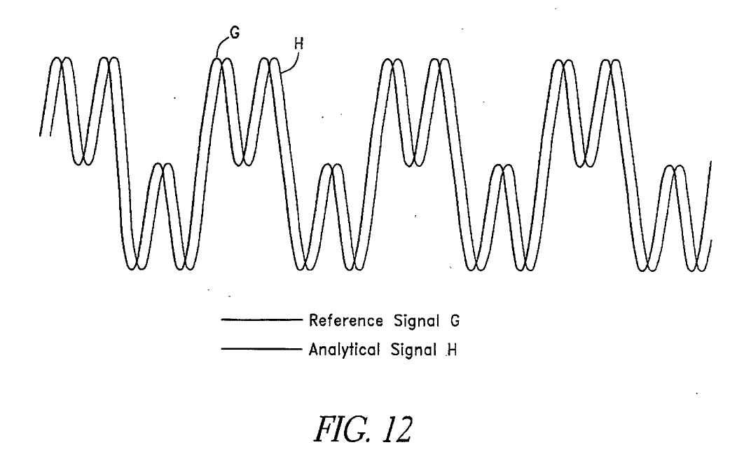

The driving frequencies (e.g., 1 Hz and 3 Hz) can be multiplexed as shown in FIG. 12. The fast (3 Hz) and slow (1 Hz) driving frequencies can be superimposed rather than sequentially implemented. During analysis, the data can be separated by frequency (using Fourier transform or other techniques) and independent measurements of phase delay at each of the driving frequencies may be calculated. Once resolved, the two sets of phase delay data are processed to determine absorbance and analyte concentration.

Additional details not necessary to repeat here may be found in U.S. Pat. No. 6,198,949, titled SOLID-STATE NON-INVASIVE INFRARED ABSORPTION SPECTROMETER FOR THE GENERATION AND CAPTURE OF THERMAL GRADIENT SPECTRA FROM LIVING TISSUE, issued Mar. 6, 2001; U.S. Pat. No. 6,161,028, titled METHOD FOR DETERMINING ANALYTE CONCENTRATION USING PERIODIC TEMPERATURE MODULATION AND PHASE DETECTION, issued Dec. 12, 2000; U.S. Pat. No. 5,877,500, titled MULTICHANNEL INFRARED DETECTOR WITH OPTICAL CONCENTRATORS FOR EACH CHANNEL, issued on Mar. 2, 1999; U.S. patent application Ser. No. 09/538,164, filed Mar. 30, 2000 and titled METHOD AND APPARATUS FOR DETERMINING ANALYTE CONCENTRATION USING PHASE AND MAGNITUDE DETECTION OF A RADIATION TRANSFER FUNCTION; U.S. Provisional Patent Application No. 60/336,404, filed Oct. 29, 2001, titled WINDOW ASSEMBLY; U.S. Provisional Patent Application No. 60/340,435, filed Dec. 12, 2001, titled CONTROL SYSTEM FOR BLOOD CONSTITUENT MONITOR; U.S. Provisional Patent Application No. 60/340,654, filed Dec. 12, 2001, titled SYSTEM AND METHOD FOR CONDUCTING AND DETECTING INFRARED RADIATION; U.S. Provisional Patent Application No. 60/336,294, filed Oct. 29, 2001, titled METHOD AND DEVICE FOR INCREASING ACCURACY OF BLOOD CONSTITUENT MEASUREMENT; and U.S. Provisional Patent Application No. 60/339,116, filed Nov. 7, 2001, titled METHOD AND APPARATUS FOR IMPROVING CLINICALLY SIGNIFICANT ACCURACY OF ANALYTE MEASUREMENTS. The entire disclosure of all of the above-mentioned patents, patent applications and publications is hereby incorporated by reference herein and made a part of this specification.

B. Whole-Blood Detection System

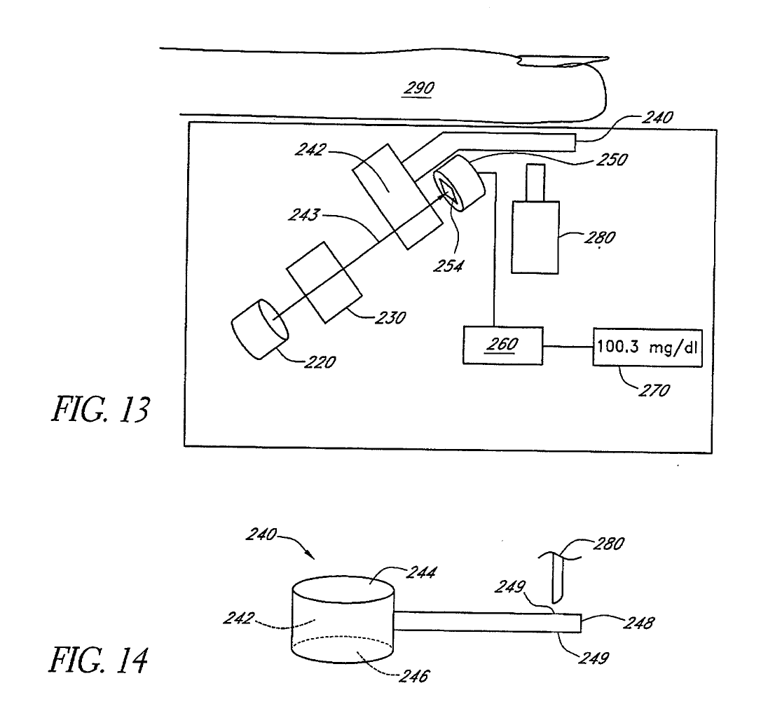

FIG. 13 is a schematic view of a reagentless whole-blood analyte detection system 200 (hereinafter “whole-blood system”) in a preferred configuration. The whole-blood system 200 may comprise a radiation source 220, a filter 230, a cuvette 240 that includes a sample cell 242, and a radiation detector 250. The whole-blood system 200 preferably also comprises a signal processor 260 and a display 270. Although a cuvette 240 is shown here, other sample elements, as described below, could also be used in the system 200. The whole-blood system 200 can also comprise a sample extractor 280, which can be used to access bodily fluid from an appendage, such as the finger 290, forearm, or any other suitable location.

As used herein, the terms “whole-blood analyte detection system” and “whole-blood system” are broad, synonymous terms and are used in their ordinary sense and refer, without limitation, to analyte detection devices which can determine the concentration of an analyte in a material sample by passing electromagnetic radiation into the sample and detecting the absorbance of the radiation by the sample. As used herein, the term “whole-blood” is a broad term and is used in its ordinary sense and refers, without limitation, to blood that has been withdrawn from a patient but that has not been otherwise processed, e.g., it has not been hemolysed, lyophilized, centrifuged, or separated in any other manner, after being removed from the patient. Whole-blood may contain amounts of other fluids, such as interstitial fluid or intracellular fluid, which may enter the sample during the withdrawal process or are naturally present in the blood. It should be understood, however, that the whole-blood system 200 disclosed herein is not limited to analysis of whole-blood, as the whole-blood system 10 may be employed to analyze other substances, such as saliva, urine, sweat, interstitial fluid, intracellular fluid, hemolysed, lyophilized, or centrifuged blood or any other organic or inorganic materials.

The whole-blood system 200 may comprise a near-patient testing system. As used herein, “near-patient testing system” is a broad term and is used in its ordinary sense, and includes, without limitation, test systems that are configured to be used where the patient is rather than exclusively in a laboratory, e.g., systems that can be used at a patient's home, in a clinic, in a hospital, or even in a mobile environment. Users of near-patient testing systems can include patients, family members of patients, clinicians, nurses, or doctors. A “near-patient testing system” could also include a “point-of-care” system.

The whole-blood system 200 may in one embodiment be configured to be operated easily by the patient or user. As such, the system 200 is preferably a portable device. As used herein, “portable” is a broad term and is used in its ordinary sense and means, without limitation, that the system 200 can be easily transported by the patient and used where convenient. For example, the system 200 is advantageously small. In one preferred embodiment, the system 200 is small enough to fit into a purse or backpack. In another embodiment, the system 200 is small enough to fit into a pants pocket. In still another embodiment, the system 200 is small enough to be held in the palm of a hand of the user.

Some of the embodiments described herein employ a sample element to hold a material sample, such as a sample of biological fluid. As used herein, “sample element” is a broad term and is used in its ordinary sense and includes, without limitation, structures that have a sample cell and at least one sample cell wall, but more generally includes any of a number of structures that can hold, support or contain a material sample and that allow electromagnetic radiation to pass through a sample held, supported or contained thereby; e.g., a cuvette, test strip, etc. As used herein, the term “disposable” when applied to a component, such as a sample element, is a broad term and is used in its ordinary sense and means, without limitation, that the component in question is used a finite number of times and then discarded. Some disposable components are used only once and then discarded. Other disposable components are used more than once and then discarded.

The radiation source 220 of the whole-blood system 200 emits electromagnetic radiation in any of a number of spectral ranges, e.g., within infrared wavelengths; in the mid-infrared wavelengths; above about 0.8 μm; between about 5.0 μm and about 20.0 μm; and/or between about 5.25 μm and about 12.0 μm. However, in other embodiments the whole-blood system 200 may employ a radiation source 220 which emits in wavelengths found anywhere from the visible spectrum through the microwave spectrum, for example anywhere from about 0.4 μm to greater than about 100 μm. In still further embodiments the radiation source emits electromagnetic radiation in wavelengths between about 3.5 μm and about 14 μm, or between about 0.8 μm and about 2.5 μm, or between about 2.5 μm and about 20 μm, or between about 20 μm and about 100 μm, or between about 6.85 μm and about 10.10 μm.

The radiation emitted from the source 220 is in one embodiment modulated at a frequency between about one-half hertz and about one hundred hertz, in another embodiment between about 2.5 hertz and about 7.5 hertz, in still another embodiment at about 50 hertz, and in yet another embodiment at about 5 hertz. With a modulated radiation source, ambient light sources, such as a flickering fluorescent lamp, can be more easily identified and rejected when analyzing the radiation incident on the detector 250. One source that is suitable for this application is produced by ION OPTICS, INC. and sold under the part number NL5LNC.

The filter 230 permits electromagnetic radiation of selected wavelengths to pass through and impinge upon the cuvette/sample element 240. Preferably, the filter 230 permits radiation at least at about the following wavelengths to pass through to the cuvette/sample element: 3.9, 4.0 μm, 4.05 μm, 4.2 μm, 4.75, 4.95 μm, 5.25 μm, 6.12 μm, 7.4 μm, 8.0 μm, 8.45 μm, 9.25 μm, 9.5 μm, 9.65 μm, 10.4 μm, 12.2 μm. In another embodiment, the filter 230 permits radiation at least at about the following wavelengths to pass through to the cuvette/sample element: 5.25 μm, 6.12 μm, 6.8 μm, 8.03 μm, 8.45 μm, 9.25 μm, 9.65 μm, 10.4 μm, 12 μm. In still another embodiment, the filter 230 permits radiation at least at about the following wavelengths to pass through to the cuvette/sample element: 6.85 μm, 6.97 μm, 7.39 μm, 8.23 μm, 8.62 μm, 9.02 μm, 9.22 μm, 9.43 μm, 9.62 μm, and 10.10 μm. The sets of wavelengths recited above correspond to specific embodiments within the scope of this disclosure. Furthermore, other subsets of the foregoing sets or other combinations of wavelengths can be selected. Finally, other sets of wavelengths can be selected within the scope of this disclosure based on cost of production, development time, availability, and other factors relating to cost, manufacturability, and time to market of the filters used to generate the selected wavelengths, and/or to reduce the total number of filters needed.

In one embodiment, the filter 230 is capable of cycling its passband among a variety of narrow spectral bands or a variety of selected wavelengths. The filter 230 may thus comprise a solid-state tunable infrared filter, such as that available from ION OPTICS INC. The filter 230 could also be implemented as a filter wheel with a plurality of fixed-passband filters mounted on the wheel, generally perpendicular to the direction of the radiation emitted by the source 220. Rotation of the filter wheel alternately presents filters that pass radiation at wavelengths that vary in accordance with the filters as they pass through the field of view of the detector 250.

The detector 250 preferably comprises a 3 mm long by 3 mm wide pyroelectric detector. Suitable examples are produced by DIAS Angewandte Sensorik GmbH of Dresden, Germany, or by BAE Systems (such as its TGS model detector). The detector 250 could alternatively comprise a thermopile, a bolometer, a silicon microbolometer, a lead-salt focal plane array, or a mercury-cadmium-telluride (MCT) detector. Whichever structure is used as the detector 250, it is desirably configured to respond to the radiation incident upon its active surface 254 to produce electrical signals that correspond to the incident radiation.

In one embodiment, the sample element comprises a cuvette 240 which in turn comprises a sample cell 242 configured to hold a sample of tissue and/or fluid (such as whole-blood, blood components, interstitial fluid, intercellular fluid, saliva, urine, sweat and/or other organic or inorganic materials) from a patient within its sample cell. The cuvette 240 is installed in the whole-blood system 200 with the sample cell 242 located at least partially in the optical path 243 between the radiation source 220 and the detector 250. Thus, when radiation is emitted from the source 220 through the filter 230 and the sample cell 242 of the cuvette 240, the detector 250 detects the radiation signal strength at the wavelength(s) of interest. Based on this signal strength, the signal processor 260 determines the degree to which the sample in the cell 242 absorbs radiation at the detected wavelength(s). The concentration of the analyte of interest is then determined from the absorption data via any suitable spectroscopic technique.

As shown in FIG. 13, the whole-blood system 200 can also comprise a sample extractor 280. As used herein, the term “sample extractor” is a broad term and is used in its ordinary sense and refers, without limitation, to any device which is suitable for drawing a sample material, such as whole-blood, other bodily fluids, or any other sample material, through the skin of a patient. In various embodiments, the sample extractor may comprise a lance, laser lance, iontophoretic sampler, gas-jet, fluid-jet or particle-jet perforator, ultrasonic enhancer (used with or without a chemical enhancer), or any other suitable device.

As shown in-FIG. 13, the sample extractor 280 could form an opening in an appendage, such as the finger 290, to make whole-blood available to the cuvette 240. It should be understood that other appendages could be used to draw the sample, including but not limited to the forearm. With some embodiments of the sample extractor 280, the user forms a tiny hole or slice through the skin, through which flows a sample of bodily fluid such as whole-blood. Where the sample extractor 280 comprises a lance (see FIG. 14), the sample extractor 280 may comprise a sharp cutting implement made of metal or other rigid materials. One suitable laser lance is the Lasette Plus® produced by Cell Robotics International, Inc. of Albuquerque, N. Mex. If a laser lance, iontophoretic sampler, gas-jet or fluid-jet perforator is used as the sample extractor 280, it could be incorporated into the whole-blood system 200 (see FIG. 13), or it could be a separate device.

Additional information on laser lances can be found in U.S. Pat. No. 5,908,416, issued Jun. 1, 1999, titled LASER DERMAL PERFORATOR; the entirety of this patent is hereby incorporated by reference herein and made a part of this specification. One suitable gas-jet, fluid-jet or particle-jet perforator is disclosed in U.S. Pat. No. 6,207,400, issued Mar. 27, 2001, titled NON- OR MINIMALLY INVASIVE MONITORING METHODS USING PARTICLE DELIVERY METHODS; the entirety of this patent is hereby incorporated by reference herein and made a part of this specification. One suitable iontophoretic sampler is disclosed in U.S. Pat. No. 6,298,254, issued Oct. 2, 2001, titled DEVICE FOR SAMPLING SUBSTANCES USING ALTERNATING POLARITY OF IONTOPHORETIC CURRENT; the entirety of this patent is hereby incorporated by reference herein and made a part of this specification. One suitable ultrasonic enhancer, and chemical enhancers suitable for use therewith, are disclosed in U.S. Pat. No. 5,458,140, titled ENHANCEMENT OF TRANSDERMAL MONITORING APPLICATIONS WITH ULTRASOUND AND CHEMICAL ENHANCERS, issued Oct. 17, 1995, the entire disclosure of which is hereby incorporated by reference and made a part of this specification.

FIG. 14 shows one embodiment of a sample element, in the form of a cuvette 240, in greater detail. The cuvette 240 further comprises a sample supply passage 248, a pierceable portion 249, a first window 244, and a second window 246, with the sample cell 242 extending between the windows 244, 246. In one embodiment, the cuvette 240 does not have a second window 246. The first window 244 (or second window 246) is one form of a sample cell wall; in other embodiments of the sample elements and cuvettes disclosed herein, any sample cell wall may be used that at least partially contains, holds or supports a material sample, such as a biological fluid sample, and which is transmissive of at least some bands of electromagnetic radiation, and which may but need not be transmissive of electromagnetic radiation in the visible range. The pierceable portion 249 is an area of the sample supply passage 248 that can be pierced by suitable embodiments of the sample extractor 280. Suitable embodiments of the sample extractor 280 can pierce the portion 249 and the appendage 290 to create a wound in the appendage 290 and to provide an inlet for the blood or other fluid from the wound to enter the cuvette 240. (The sample extractor 280 is shown on the opposite side of the sample element in FIG. 14, as compared to FIG. 13, as it may pierce the portion 249 from either side.)

The windows 244, 246 are preferably optically transmissive in the range of electromagnetic radiation that is emitted by the source 220, or that is permitted to pass through the filter 230. In one embodiment, the material that makes up the windows 244, 246 is completely transmissive, i.e., it does not absorb any of the electromagnetic radiation from the source 220 and filter 230 that is incident upon it. In another embodiment, the material of the windows 244, 246 has some absorption in the electromagnetic range of interest, but its absorption is negligible. In yet another embodiment, the absorption of the material of the windows 244, 246 is not negligible, but it is known and stable for a relatively long period of time. In another embodiment, the absorption of the windows 244, 246 is stable for only a relatively short period of time, but the whole-blood system 200 is configured to observe the absorption of the material and eliminate it from the analyte measurement before the material properties can change measurably.

The windows 244, 246 are made of polypropylene in one embodiment. In another embodiment, the windows 244, 246 are made of polyethylene. Polyethylene and polypropylene are materials having particularly advantageous properties for handling and manufacturing, as is known in the art. Also, polypropylene can be arranged in a number of structures, e.g., isotactic, atactic and syndiotactic, which may enhance the flow characteristics of the sample in the sample element. Preferably the windows 244, 246 are made of durable and easily manufactureable materials, such as the above-mentioned polypropylene or polyethylene, or silicon or any other suitable material. The windows 244, 246 can be made of any suitable polymer, which can be isotactic, atactic or syndiotactic in structure.

The distance between the windows 244, 246 comprises an optical pathlength and can be between about 1 μm and about 100 μm. In one embodiment, the optical pathlength is between about 10 μm and about 40 μm, or between about 25 μm and about 60 μm, or between about 30 μm and about 50 μm. In still another embodiment, the optical pathlength is about 25 μm. The transverse size of each of the windows 244, 246 is preferably about equal to the size of the detector 250. In one embodiment, the windows are round with a diameter of about 3 mm. In this embodiment, where the optical pathlength is about 25 μm the volume of the sample cell 242 is about 0.177 μL. In one embodiment, the length of the sample supply passage 248 is about 6 mm, the height of the sample supply passage 248 is about 1 mm, and the thickness of the sample supply passage 248 is about equal to the thickness of the sample cell, e.g., 25 μm. The volume of the sample supply passage is about 0.150 μL. Thus, the total volume of the cuvette 240 in one embodiment is about 0.327 μL. Of course, the volume of the cuvette 240/sample cell 242/etc. can vary, depending on many variables, such as the size and sensitivity of the detectors 250, the intensity of the radiation emitted by the source 220, the expected flow properties of the sample, and whether flow enhancers (discussed below) are incorporated into the cuvette 240. The transport of fluid to the sample cell 242 is achieved preferably through capillary action, but may also be achieved through wicking, or a combination of wicking and capillary action.

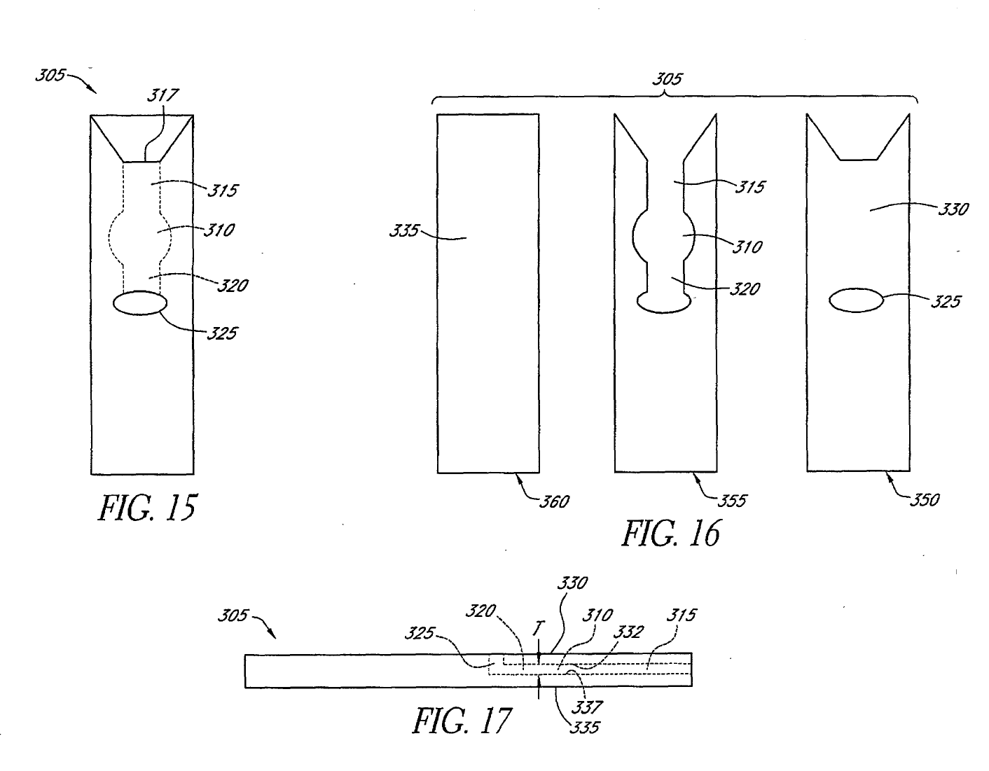

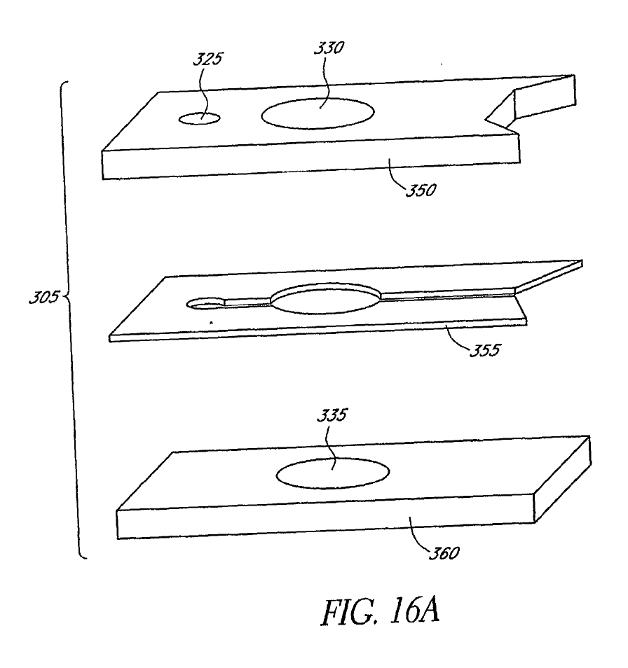

FIGS. 15-17 depict another embodiment of a cuvette 305 that could be used in connection with the whole-blood system 200. The cuvette 305 comprises a sample cell 310, a sample supply passage 315, an air vent passage 320, and a vent 325. As best seen in FIGS. 16, 16A and 17, the cuvette also comprises a first sample cell window 330 having an inner side 332, and a second sample cell window 335 having an inner side 337. As discussed above, the window(s) 330/335 in some embodiments also comprise sample cell wall(s). The cuvette 305 also comprises an opening 317 at the end of the sample supply passage 315 opposite the sample cell 310. The cuvette 305 is preferably about ¼-⅛ inch wide and about ¾ inch long; however, other dimensions are possible while still achieving the advantages of the cuvette 305.

The sample cell 310 is defined between the inner side 332 of the first sample cell window 330 and the inner side 337 of the second sample cell window 335. The perpendicular distance T between the two inner sides 332, 337 comprises an optical pathlength that can be between about 1 μm and about 1.22 mm. The optical pathlength can alternatively be between about 1 μm and about 100 μm. The optical pathlength could still alternatively be about 80 μm, but is preferably between about 10 μm and about 50 μm. In another embodiment, the optical pathlength is about 25 μm. The windows 330, 335 are preferably formed from any of the materials discussed above as possessing sufficient radiation transmissivity. The thickness of each window is preferably as small as possible without overly weakening the sample cell 310 or cuvette 305.

Once a wound is made in the appendage 290, the opening 317 of the sample supply passage 315 of the cuvette 305 is placed in contact with the fluid that flows from the wound. In another embodiment, the sample is obtained without creating a wound, e.g. as is done with a saliva sample. In that case, the opening 317 of the sample supply passage 315 of the cuvette 305 is placed in contact with the fluid obtained without creating a wound. The fluid is then transported through the sample supply passage 315 and into the sample cell 310 via capillary action. The air vent passage 320 improves the capillary action by preventing the buildup of air pressure within the cuvette and allowing the blood to displace the air as the blood flows therein.

Other mechanisms may be employed to transport the sample to the sample cell 310. For example, wicking could be used by providing a wicking material in at least a portion of the sample supply passage 315. In another variation, wicking and capillary action could be used together to transport the sample to the sample cell 310. Membranes could also be positioned within the sample supply passage 315 to move the blood while at the same time filtering out components that might complicate the optical measurement performed by the whole-blood system 200.

FIGS. 16 and 16A depict one approach to constructing the cuvette 305. In this approach, the cuvette 305 comprises a first layer 350, a second layer 355, and a third layer 360. The second layer 355 is positioned between the first layer 350 and the third layer 360. The first layer 350 forms the first sample cell window 330 and the vent 325. As mentioned above, the vent 325 provides an escape for the air that is in the sample cell 310. While the vent 325 is shown on the first layer 350, it could also be positioned on the third layer 360, or could be a cutout in the second layer, and would then be located between the first layer 360 and the third layer 360 The third layer 360 forms the second sample cell window 335.

The second layer 355 may be formed entirely of an adhesive that joins the first and third layers 350, 360. In other embodiments, the second layer may be formed from similar materials as the first and third layers, or any other suitable material. The second layer 355 may also be formed as a carrier with an adhesive deposited on both sides thereof. The second layer 355 forms the sample supply passage 315, the air vent passage 320, and the sample cell 310. The thickness of the second layer 355 can be between about 1 μm and about 1.22 mm. This thickness can alternatively be between about 1 μm and about 100 μm. This thickness could alternatively be about 80 μm, but is preferably between about 10 μm and about 50 μm. In another embodiment, the second layer thickness is about 25 μm.

In other embodiments, the second layer 355 can be constructed as an adhesive film having a cutout portion to define the passages 315, 320, or as a cutout surrounded by adhesive.

Further information can be found in U.S. patent application Ser. No. 10/055,875, filed Jan. 21, 2002, titled REAGENT-LESS WHOLE-BLOOD GLUCOSE METER. The entire contents of this patent application are hereby incorporated by reference herein and made a part of this specification.

### Method and Apparatus for Improving the Accuracy of an Alternative Site Analyte Concentration Measurement

The methods and apparatus described in the previous section titled OVERVIEW OF ANALYTE DETECTION SYSTEMS and illustrated in FIGS. 1-17 can generally be used with any number of methods and apparatuses for determining analyte concentrations, and more specifically can be used with the methods and apparatuses for allowing a patient to determine analyte concentrations at less innervated testing sites, such as, for example, at an AST, as disclosed herein. Traditional blood samples for Self Monitoring of Blood Glucose (“SMBG”) are taken from a finger with a lance. Tests have shown that finger blood glucose levels correlate highly to systemic blood glucose values. Fingertips are highly vascularized and therefore offer an ideal site from which to draw relatively large (1-3 uL) drops of blood for SMBG analysis. Fingertips are also highly innervated, providing good tactile sense which unfortunately makes lancing to obtain the blood sample painful.

Alternative site testing can be conducted at sites less highly innervated. Reduced innervation produces less or in some cases no pain. However, such alternative sites typically are less vascularized and therefore provide smaller blood samples (0.3-1 uL). Recently, SMBG instruments using lower blood volumes have made alternative site testing popular. A new class of SMBG instruments which are capable of measuring glucose levels with no blood withdrawal at all (e.g. noninvasive) can also be employed to make alternative site measurements.

One of the most popular alternative sites is the forearm. The concentrations of blood constituents such as glucose measured at alternative sites, particularly the forearm, can exhibit a time lag relative to simultaneous concentration measurements taken at the finger. The time lag varies according to the individual; however, 10 minutes is a typical value for this time lag. If a person's blood glucose level is changing rapidly, for instance 3 mg/dL per min., a time lag of 10 minutes will produce an erroneous alternative-site reading of 30 mg/dL.

Regardless of where glucose measurements are taken, rapid changes in blood glucose concentration do not always exist. Most of the time the glucose level is steady or is changing slowly (i.e. the magnitude of rate of change is less than 1 mg/dL per minute). However, after an individual eats, the blood glucose concentration may rise rapidly. Similarly, blood glucose concentration may fall rapidly after an individual engages in strenuous exercise. The instantaneous value of the blood glucose rate of change almost always falls between −3 and +3 mg/dL per minute, typically between −2.5 and +2.5 mg/dL per minute, depending on the type and level of the individual's activity.

Disclosed herein are methods and apparatus for improving the accuracy of a blood-constituent measurement and/or of a blood-constituent measurement device. The disclosed methods and apparatus are further suitable for improving the accuracy of an alternative-site (“AST”) blood-constituent measurement device. As used herein, the term “alternative-site blood-constituent measurement device” (“AST device”) is a broad term and is used in its ordinary sense and refers, without limitation, to analyte detection devices which are capable of (but need not be restricted to) measuring the concentration of one or more blood constituents, such as glucose or any other analyte, at an alternative site. As used herein, the term “alternative site” is a broad term and is used in its ordinary sense and refers, without limitation, to any measurement location on the body other than the fingers, such as the forearm or abdomen.

Although much of the discussion herein takes place within the exemplary environment of determining the concentration of glucose within a subject's blood, it should be noted that the methods and apparatus disclosed herein are not limited to blood glucose concentration. Rather, the disclosed methods and apparatus may be employed in the measurement of a wide variety of analytes, including but not limited to alcohol, urea, or any other analyte disclosed herein, within a wide variety of bodily fluids or tissues, including but not limited to interstitial fluid, intercellular fluid, saliva, urine, tissue biopsies, or any other fluids or sample types disclosed herein. Therefore, any reference herein to blood, glucose, or blood constituents should be construed as exemplary and not limiting. A blood constituent is one form of analyte, but the methods and apparatus disclosed herein can apply to any analyte.

As discussed below, various embodiments of an AST correction method generally comprise: (1) calculating or estimating the rate of change of concentration of an analyte or blood constituent, such as glucose; and/or (2) correcting information provided to an AST device user based on the observed rate of change. Apparatuses for carrying out these methods are also disclosed. The various methods described and illustrated herein may be done by device, person, or a combination of device and person.

In one method, the AST correction method is used to calibrate the AST device. The AST device and correction method may be used in numerous applications, including, but not limited to, monitoring blood glucose levels, detecting and analyzing changes and trends in blood glucose levels, and forecasting blood glucose levels. The AST device and correction method may be used in the context of blood glucose measurements obtained invasively or noninvasively or through a combination of both invasive and noninvasive measures.

In another method, the AST device utilizes the rate of glucose-concentration change to determine the accuracy of AST blood glucose measurements. In one embodiment, there is provided a method for determining the rate of change of a patient's blood glucose concentration value and for determining whether or not this rate of change is large enough to produce significant errors in AST SMBG measurements. In another embodiment, the rate of change information is used to correct for errors that may be present in AST measurements. In yet another embodiment, an AST device is configured to either warn of possible errors or automatically correct for the errors after determining that there is a large rate of change in the blood glucose concentration which will produce significant errors in AST SMBG measurements.

Any method which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the method(s) in question.

A. Timing Circuit

The methods disclosed herein may advantageously utilize or be executed by or with an AST device which incorporates a timing circuit. For example, the AST device may comprise various embodiments of the noninvasive system 10 disclosed above, with a timing circuit accessible by the signal processor 74 (see FIG. 7). Alternatively, the AST device may comprise various embodiments of the whole-blood system 200 disclosed above, with a timing circuit accessible by the signal processor 260 (see FIG. 13). In further alternatives, the AST device can comprise any suitable AST device with a timing circuit.

As used herein, the term “timing circuit” is a broad term and is used in its ordinary sense and refers, without limitation, to any device that is capable of tracking or calculating intervals of time. The presence of a timing circuit allows the AST device to determine time intervals between glucose measurements for rate change calculations. In one embodiment, the timing circuit comprises a real-time clock. As used herein, the term “real-time clock” is a broad term and is used in its ordinary sense and refers, without limitation, to any device that is capable of tracking or calculating intervals of time and keeping the time of day, the day of week, the day of month, the month and/or the year.

Alternatively, the timing circuit can be any timing system or mechanism either attached or detached from the AST device. In one embodiment, the timing circuit is an internal clock which time-stamps glucose measurements. In another embodiment, the timing circuit comprises a user-interface whereby the user is able to manually enter time information into the AST device. For example, the user may enter time information based on an external clock, such as, for example, a wristwatch or a wall clock. In yet another embodiment, time information is transmitted from external clocks to the AST device via signals, such as, for example, radio signals, infrared signals, etc.

With continued reference to FIGS. 7 and 13, it should be further noted that in executing the methods disclosed herein, computations may be performed by the signal processor 74 on data stored within memory accessible thereby (where the method is executed by the noninvasive system 10), or by the signal processor 260 on data stored within memory accessible thereby (where the method is executed by the whole-blood system 200). Any of the methods disclosed herein may reside in memory as a data-processing algorithm or program instructions executable by the processor 74 of the noninvasive system 10, by the processor 260 of the whole-blood system 200, or by processing circuit or hardware of any suitable AST device. In one embodiment, the processing circuit works in conjunction with a module that is executable by the processing circuit. In one embodiment, the module can comprise one or more electrical devices, mechanical devices, components thereof, instructions for operation, combinations thereof, etc.

B. Determination Of Rate Of Analyte-Concentration Change

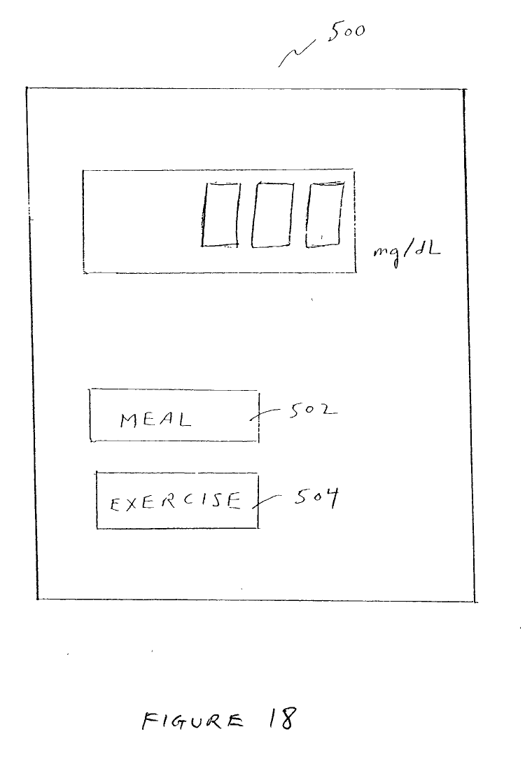

Various methods may be employed to determine the rate of analyte-concentration change. In one method, the user inputs information into the AST device. Such information could include “Meal” to indicate that the user is about to eat or has eaten a meal, or “Exercise” to indicate that the user is about to engage in or has engaged in strenuous physical activity. To support this method the AST device 500 could include a Meal button 502 and/or an Exercise button 504 (see FIG. 18) for convenient input of relevant information. However, any suitable structure may be employed to facilitate this data entry.

In one embodiment, the AST device estimates the rate of analyte-concentration change by assigning a predetermined rate of analyte-concentration change to a given type of input information. For example, the AST device may assign a high positive value to the rate of change, such as that often observed after a meal, if the user pushes the Meal button, or a high negative rate of change, such as that often observed after exercise, if the user pushes the Exercise button. The actual numerical value of each predetermined rate of change would preferably be calibrated for the device user.

The AST device may advantageously be configured to time stamp the input information based on the output of the real-time clock. In response to the input information, the AST device notes or records within its memory that the absolute value of the rate of change of analyte/glucose concentration will be higher than normal for a given duration, for example, 1 hour, 1.5 hours, or 2 hours after eating or after exercise.

In another method of determining the rate of analyte/glucose-concentration change, the AST device is configured to record and time stamp the analyte-concentration measurements. As one approach, analyte/glucose-concentration measurements may be recorded at two different times (t₁ and t₂, for example), and then rate of change may be computed as (concentration at t₂−concentration at t₁)/(t₂−t₁). It should be noted that (concentration at t₂−concentration at t₁) may comprise samples from a continuously taken measurement.

In the absence of a real-time clock, t₁ and t₂ may be limited to being two successive times, such as Monday 10:20:00 for t₁ and Monday 10:30:00 for t₂. In contrast, the utilization of a real-time clock also allows one to monitor trends in concentration and trends in rates of change. For example, with a real-time clock the AST device may be operated or configured to take measurements at times t₁, t₂, and t₃, and then compute rates of change between t₁ and t₂, between t₂ and t₃, and/or between t₁ and t₃. Further, rates of change may be computed for the aforementioned times (t₁, t₂, and t₃) on day 1 and the corresponding times (t₁, t₂, and t₃) on day 2, and so on.

In one embodiment, measurements may be taken every 10 minutes; however, the frequency of such measurements can vary. More frequent measurements are helpful for detecting and correcting for rapid rates of analyte/glucose-concentration change. In addition, more frequent measurements provide a larger number of sample measurements with which to determine if there exists a consistency or trend among the measured values. Similarly, a higher frequency of measurements facilitates the process of determining whether a given computed rate of change is an outlier that should be disregarded.

It should be noted, however, that a higher frequency of measurements not only requires more measurements that need to be taken in a given period of time, but it also requires more memory to store the measurement values, both of which result in the use of more system resources. Thus, there is a tradeoff between measurement rate and available system resources. In an alternative embodiment, which can be implemented when the availability of system resources is not a limiting factor, the AST device takes continuous measurements.

C. Correcting Information Provided To User

Various methods may be employed to correct the information provided to the AST device user by the AST device.

1. Display a Warning

In one method, the AST device displays and/or sounds a warning that the absolute value of the rate of change of analyte/glucose-concentration is high and that AST measurements may be in error. Instead of or in addition to such a warning, the AST device could prompt the user to make a fingertip or on-site measurement, either invasively or noninvasively.

2. External Model

A further method of correcting the information provided to the user involves determining the clinical significance of an error in an AST measurement. Preferably, the AST device uses an external model, such as, for example, a variation of the Clarke Error Grid or the like, to assess the clinical significance of a computed AST error.

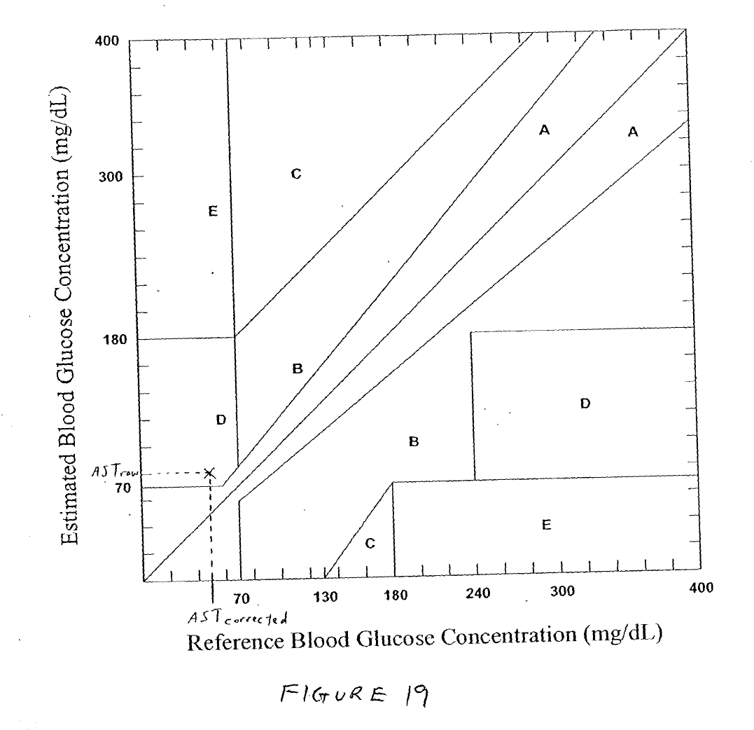

A rate of change of glucose concentration is determined by any of the methods disclosed above. An AST error is computed based on the rate of change and a typical AST time lag, for example 10 minutes. An AST measurement is taken yielding a raw (i.e. uncorrected) AST glucose value AST_{raw}. In addition, a corrected AST glucose value AST_{corrected} is computed using the AST error as discussed above. The significance of the error is assessed by using an external model. With reference to FIG. 19, in one embodiment, the significance of the error is determined on a variation of the Clarke error grid by plotting the intersection of the glucose values: AST_{raw} on the Estimated Blood Glucose Concentration (vertical) axis and AST_{corrected} on the Reference Blood Glucose Concentration (horizontal) axis.

With continued reference to FIG. 19, the significance of the error is determined by the zone in which the resulting data point lies. Zones A and B represent values where the errors in AST measurements are considered clinically acceptable. Zone C represents values that would result in an unnecessary correction of acceptable glucose. Zone D represents values that would result in a dangerous failure to detect and treat. Zone E represents values that would lead to treatment opposite of what clinical accuracy would call for. If the variation of the Clarke error grid analysis yields an AST measurement error represented by zones C, D, or E, the AST device warns the user to measure glucose concentration at an on-site location, such as the finger or finger-tip. Alternatively, the AST device could so warn the user if the variation of the Clarke error grid analysis yields a zone B, C, D or E error, or a zone D or E error.

For example, assume the rate of change is measured or known to be high, such as −3 mg/dL per min due to strenuous exercise. The AST device computes a possible AST error of −30 mg/dL based on a typical 10 minute lag. An AST measurement is made and a value of 80 mg/dL is found. The AST device, referring to a variation of the Clarke error grid, determines that the value of 80 mg/dL less the potential lag error of −30 mg/dL yields a value of 50 mg/dL, which yields a zone D error as shown in FIG. 19. In this case the AST device warns the user to make another measurement using an on-site location like the finger or fingertip.

3. Automatic Correction

In another method, the AST device advantageously automatically corrects an AST measurement based on rate of change information. To implement the automatic correction process, the device develops a user specific correction factor via individual calibration. Although it is possible that several users may share the same correction factor, it is preferred to develop a distinct correction factor for each user.

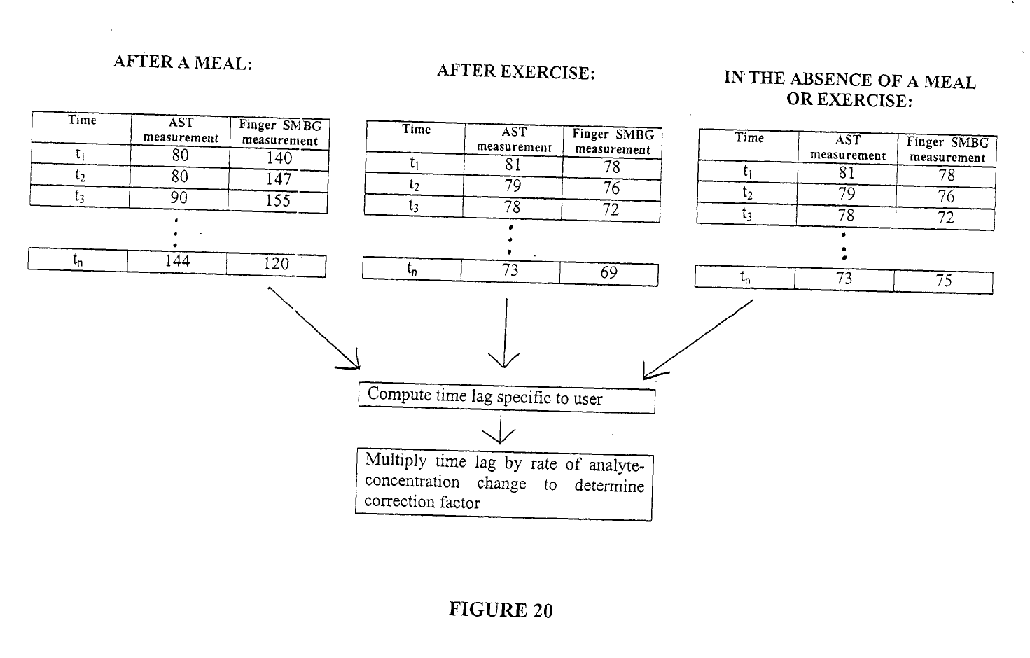

In one embodiment, illustrated in FIG. 20, the correction factor is developed by making repeated AST and on-site analyte concentration measurements under conditions selected to capture a range of analyte change rates. In the embodiment shown in FIG. 20, this could be done by using the AST device to measure and record the concentration of an analyte, for example glucose, after a meal, after exercise, and/or in the absence of either of these activities. Methods and conditions under which glucose measurements are taken for individual calibration of the AST device include but are not limited to glucose tolerance tests and/or glucose measurements taken during and/or after strenuous physical activity. In alternative embodiments, repeated AST determinations are made in conjunction with withdrawal of blood samples and/or noninvasive measurements, either with or without traditional finger/fingertip SMBG determinations.

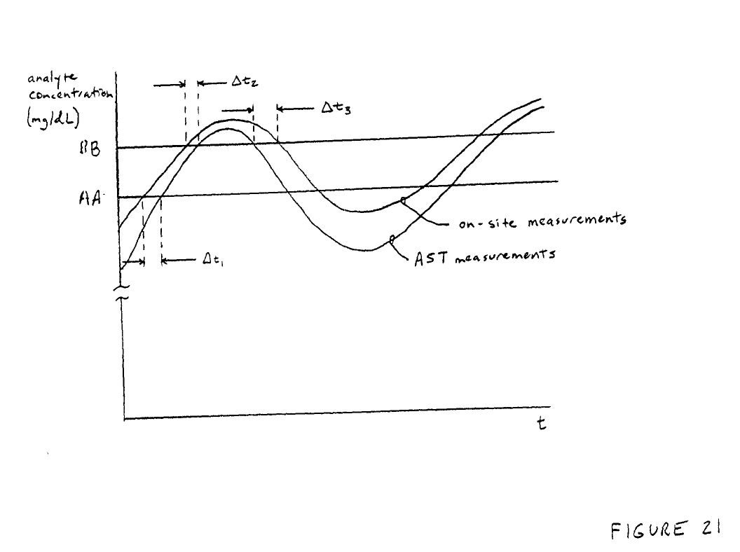

Once a set of user-specific information relating to analyte concentration, measurement location, and/or measurement time is recorded by or entered into the AST device, the device computes at last one AST time lag specific to that individual. In one embodiment, this is done by plotting the observed analyte concentrations vs. time, for on-site and AST measurements (see FIG. 21). At one or more selected analyte concentrations AA, BB, etc., one or more time lags Δt₁, Δt₂, Δt₃ can be calculated from the distance along the time-axis between the respective intercepts of the on-site and AST curves with a horizontal line representing the concentration in question. The computed time lags can be averaged to generate an overall time lag specific to the user. In one embodiment, separate user-specific time lags may be calculated in this manner for “Meal,” “Exercise,” and/or “No Meal/Exercise” conditions by relying only on the data corresponding to the condition in question. As discussed above with reference to FIG. 18, the AST device may advantageously be configured to accept user input of the present condition and select the appropriate time lag based on the selected condition. It is contemplated that the data collection/recording and the time lag calculations described herein may be done manually or by the AST device, or by a combination of manual and device-reliant techniques.

With the time lag thus determined, the AST device multiplies the lag by the rate of analyte change determined by any of the methods disclosed herein to calculate a correction factor. The correction factor is either added to or subtracted from the AST measurement to correct it and make it consistent with the on-site value. In other embodiments, some or all of these calculations may be performed manually, instead of or in addition to machine calculation.

For example, suppose that the user or AST device has information from an individual consisting of simultaneous on-site and AST analyte-concentration measurements through a period of rapidly changing analyte concentration, such as after a meal and during exercise. From this information the AST device or user computes that the AST vs. on-site lag in that individual is 11 min. An AST measurement is made and a value of 100 mg/dL found. At the time of the AST measurement the user or AST device determines that the analyte rate of change is +1 mg/dL/min. The user or AST device then computes a correction factor (11 min*1 mg/dL per min=11 mg/dL). This factor is added to the measured value of 100 mg/dL and the AST device may advantageously display the corrected value of 111 mg/dL.

In another embodiment, the AST device increases the accuracy of AST measurements by using time data from a real-time clock to extrapolate AST measurements based on the user's AST time lag. For example, suppose that a user has just eaten a meal and that his/her AST time lag is known or has previously been determined to be 17 minutes. Given that the user's AST time lag is 17 min., an AST measurement at t=17 min. provides an accurate estimate of an on-site measurement at t=0 min. In one embodiment, the AST device takes blood-glucose measurements every 10 minutes. Suppose that the user's AST blood-glucose is 80 mg/dL at t=0 min. and 120 mg/dL at t=10 min. Under one approach, the AST device uses linear extrapolation to determine what AST blood-glucose will be at t=17 min. Here, the rate of AST blood-glucose change can be calculated as (120−80 mg/dL)/(10−0 min.), which yields 4 mg/dL/minute. Knowing the rate of change and time lag, the AST device can extrapolate what the AST blood-glucose level will be at t=17 min. as follows:

Blood-glucose=[rate of change*(time interval between t=17 min. and initial time)]+(AST glucose level at initial time).

Here, blood-glucose=[4 mg/dL/minute*(17−0 minutes)]+80 mg/dL, or blood-glucose=[4 mg/dL/minute*(17−10) minutes]+120 mg/dL. Both sets of calculations yield a glucose concentration of 148 mg/dL, as opposed to a value of 80 mg/dL which does not take into account the user's AST time lag. In another embodiment, the AST device uses curve-fitting and/or other non-linear extrapolation techniques to estimate on-site analyte-concentration values based on AST measurements and the AST time lag.

4. Outlier Rejection and Forecasting

In another method, the AST device corrects the information provided to the AST device user by screening the measured concentrations of a blood constituent, such as glucose, and/or the calculated rates of concentration change in order to remove outlier concentration and/or outlier rate values from consideration while monitoring rates of change and/or communicating results to a user. As used herein, the term “outlier value” is a broad term and is used in its ordinary sense and refers, without limitation, to a value that is far removed from other values in a set of data. Consequently, an outlier value may create an extreme deviation from the expected or average value of a set of data. For example, calculating the rate of change using an outlier blood-constituent value will likely result in an erroneous rate of change value, which may in turn decrease the accuracy of alternate site blood-constituent measurements.

In one embodiment, the AST device rejects outlier values (i.e. measurement values or rate of change calculations based on measurement values) independently of the AST correction methods described above. In another embodiment, the AST device advantageously applies the AST correction methods and rejects outlier values, either before or after applying the AST correction method.

In the examples illustrated herein, the analyte concentration (“ACₙ”) measurements and rate of analyte-concentration change (“RCₙ”) calculations are done on an intermittent basis. In other embodiment, however, the AST device takes continuous ACₙ measurements and/or calculates RCₙ values on a continuous basis.

For example, in one embodiment, the AST device stores ACₙ values over a period of five hours and time-stamps each AST measurement. The duration of the period-of-time or time-episode during which the AST device measures, time-stamps, and stores ACₙ values is determined and/or adjusted by the AST device or user. In another embodiment, the time-episode and/or the ACₙ values measured during the time-episode are labeled and/or categorized by the conditions (e.g. “Meal,” “Exercise,” and/or “No Meal/Exercise”) present during the time-episode, as determined by the AST device or user. In the example illustrated herein, the AST device measures, time-stamps, and stores thirty ACₙ values (ACₙ₋₁, ACₙ₋₂, . . . ACₙ₋₃₀) over a period of five hours. Preferably, these AST measurements are taken at equally spaced time intervals. In the present example, the AST device measures ACₙ values every 10 minutes.

In a preferred embodiment, the AST device uses statistical analysis to calculate a mean and standard deviation for the set of ACₙ data for a given time-episode. Typically, for a normal or Gaussian distribution of data, one standard deviation away from the mean accounts for approximately 68 percent of the values in the data set. Two standard deviations away from the mean account for approximately 95 percent of the values in the data set. Three standard deviations account for approximately 99 percent of the values in the data set. For each time-episode, the user and/or AST device uses the mean ACₙ and standard deviation values to define a permissible range of values to be included in the measurement history (ACₙ₋₁, ACₙ₋₂, etc.). In other embodiments, different statistical approach methods and/or user-specific parameters, including but not limited to maximum and/or minimum analyte concentrations and/or rates of analyte-concentration change, as determined, for example, by glucose tolerance tests, may be used to define a permissible range of values to be included in the measurement history.

In the example illustrated herein, the AST device defines the permissible range of ACₙ values as two standard deviations away from the mean ACₙ value. For each ACₙ measurement, the AST device determines whether the ACₙ value falls within two standard deviations of the mean ACₙ value. If the ACₙ value falls within the permissible range, the AST device stores this ACₙ value and/or uses it for other determinations, such as calculation of the rate of analyte-concentration change. If the ACₙ value falls outside of the permissible range, the AST device: does not record the ACₙ value in the measurement history; does not report the outlier measurement to the user; takes another ACₙ measurement; warns the user to make another measurement using an on-site location like the finger or fingertip; and/or rejects any rate of change calculations done based on the outlier measurement.

Similarly, in another embodiment, the AST device calculates and RCₙ data for periods of time defined and/or categorized by the AST device and/or user. For example, in one embodiment, the AST device calculates and stores RC_{HOUR} data for one-hour time-episodes throughout a twenty-four time period. More specifically, for a given one-hour time-episode, the AST device uses the corresponding ACₙ data (i.e. ACₙ₋₁, ACₙ₋₂, etc.) to calculate a RCₙ for each pair of successive ACₙ values (RCₙ₋₁, RCₙ₋₂, etc.). The AST device then calculates the mean value (RCₕₒᵤᵣ) for the RCₙ data set. The AST device performs this calculation twenty-four times in a day with each of the twenty-four RCₕₒᵤᵣ values corresponding to a one-hour time-episode. Next, the AST device calculates the mean and standard deviation for the data set containing these twenty-four RCₕₒᵤᵣ values.

In the example illustrated herein, the AST device defines the permissible range of RCₕₒᵤᵣ values as being within two standard deviations away from the mean RCₕₒᵤᵣ value. Analogous to the example above which illustrates the rejection of outlier ACₙ values, if the RCₙ value falls within the permissible range (i.e. two standard deviations from the mean RCₕₒᵤᵣ value), the AST device stores this RCₙ value and/or uses it for calculations based on RCₙ values, including but not limited to the correction factor for an AST measurement. If the RCₙ value falls outside of the permissible range, the AST device: does not record the RCₙ value in the measurement history; does not report the outlier RCₙ value to the user; takes another ACₙ measurement with which to calculate another RCₙ value; warns the user to make another ACₙ measurement using an on-site location like the finger or fingertip; and/or rejects any calculations based on the outlier RCₙ value.

In another method, the AST device is advantageously configured to forecast future ACₙ values (ACₙ₊₁, ACₙ₊₂, etc.) and/or RCₙ values (RCₙ₊₁, RCₙ₊₂, etc.). The AST device extrapolates future ACₙ and/or RCₙ values by analyzing past values of ACₙ (i.e. ACₙ₋₁, ACₙ₋₂, etc.) and RCₙ (i.e. RCₙ₋₁, RCₙ₋₂, etc.) in the measurement history. The AST device creates or maintains sets of data based on: time of day; day of the week; and/or conditions present during a time-episode.

In a preferred embodiment, the AST device uses statistical analysis to calculate the mean and standard deviation for sets of data in the measurement histories of ACₙ and RCₙ. For example, in one illustrative embodiment, the AST device forecasts the rate of glucose-concentration change for Thursday 8:00:00 by first creating a subset (i.e. ACₙ₋₁, ACₙ₋₂, and ACₙ₋₃) of the measurement history corresponding to AST glucose concentration measurements taken on Monday 8:00:00, Tuesday 8:00:00, and Wednesday 8:00:00 during the same week. The AST device then calculates past rates of glucose-concentration change (i.e. RCₙ₋₁, RCₙ₋₂, and RCₙ₋₃) based on the value of ACₙ₋₁, ACₙ₋₂, and ACₙ₋₃. Next, the AST device calculates a mean value (“RCₘₑₐₙ”) and standard deviation for the subset of data consisting of RCₙ₋₁, RCₙ₋₂, and RCₙ₋₃. In the example illustrated herein, the AST device's forecasted RCₙ value for Thursday 8:00:00 is equal to RCₘₑₐₙ.

In the embodiment illustrated above, the AST device forecasts the future value of RCₙ by calculating the average and standard deviation of past values of RCₙ. In other embodiments, different statistical methods and/or curve-fitting techniques may be implemented on subsets and/or entire sets of past ACₙ and/or RCₙ values to extrapolate future ACₙ and/or RCₙ values.

D. Adjustment Factor and Discrete Time Analyte Measurements

In one aspect of the present invention, various methods may be employed to ensure that a user's blood-analyte concentration level returns to or is maintained at basal or normal levels. These methods generally involve: (1) having a physician or clinical practitioner determine at least one adjustment factor for the patient; and (2) utilizing an AST device that advises the patient to take the appropriate dose of treatment/medicine at certain times of the day. In one embodiment, the AST device utilizes the clinically determined adjustment factor to perform the user/patient advisory function described herein. As used herein, treatment/medicine generally refers to anything that the patient consumes or injects into him or herself in order to control his/her blood-analyte concentration. For example, in the context of controlling a patient's blood-glucose concentration, the treatment/medicine can comprise one or more of insulin, glucose, food, or any other medicine or consumable that affects the patient's blood-glucose levels.

In one embodiment, a physician, clinical practitioner, or other qualified healthcare professional (hereinafter collectively referred to as “physician”) determines the adjustment factor for a certain treatment/medicine, such as, for example, insulin, that is needed to return the patient's blood-glucose concentration to basal or normal levels. For example, the adjustment factor can be expressed as units of insulin needed per gram of carbohydrate consumed by the user. In one embodiment, in the context of using insulin to regulate blood-glucose levels, the adjustment factor comprises the Insulin Adjustment Factor (“IAF”). It should be noted that the adjustment factor is not determined by the AST device, but is instead determined by a physician. It should also be noted that the adjustment factor can vary from individual to individual.

In one embodiment, the present method of having the AST device advise the user to take a certain dose of treatment/medicine in order to return the user's blood-analyte levels to the target level (i.e. the normal or basal level) is incorporated into the AST device. However, the present method is not central to or required for the operation of the AST device. In one embodiment, the AST device has an on/off switch with which to activate/deactivate the user advisory function. It will generally be up to the user's physician to determine whether or not to activate the user advisory function of the AST device. If the physician decides that it would be desirable to activate the user advisory function, then the physician will determine the user's adjustment factor and enter this number into the AST device via a data-input or user-interface.

In one embodiment, the AST device includes a data-input interface with which the physician can input the target analyte level. Here, the AST device calculates the appropriate dose of treatment/medicine that the user should take based on the user's current, corrected AST blood-analyte measurement, the user's target analyte level, and the user's adjustment factor. The AST device then advises the user to take the calculated dose of treatment/medicine. For example, in the context of managing blood-glucose levels where the user is undergoing a hyperglycemic episode, the AST device will advise the user to take a certain dosage of insulin in order to return the blood-glucose to a basal or resting levels.

In one embodiment, the AST device calculates the appropriate dose of treatment/medicine by utilizing a computer-based algorithm, such as, for example, a neural network system. AST device training signals that include variables, such as, for example, the current analyte concentration, the target analyte concentration, the adjustment factor, the treatment/medicine dosage, etc., are fed into the neural network. In one embodiment, the neural network system provides or estimates appropriate doses of treatment/medicine for each measured blood-analyte measurement based on current input information and historical data values. It should be noted that any computer-based algorithm or training system known in the art may be implemented to program the AST device to notify the user of the appropriate treatment/medicine dosage.

In another aspect of the present invention, the AST device, which includes a real-time clock, reminds the user to take blood-analyte measurements several times throughout the day. In one preferred embodiment, the AST device reminds the user to take measurements during wake hours only. The real-time clock allows for time data and time interval data to be stored automatically. In one embodiment, in the exemplary context of blood-glucose management, the user can take a finger-stick glucose measurement in response to the reminder by the AST device. Here, the user can enter glucose measurement data into the AST device via a data-input interface or via any other appropriate data storage means. Glucose measurements can be done on the meter (i.e. the AST device) as well as manually. In another embodiment, the user can simply utilize the AST device itself to measure blood-glucose.

Over time, through repeated measurements, the AST device allows the physician to compile trends in the user's blood-analyte levels during the user's wake hours. In one embodiment, the AST device will remind the user to take analyte measurements 6-8 times during the user's wake hours. For example, in one embodiment, the device reminds the user to take analyte measurements when he/she: wakes up; eats breakfast; before lunch; after lunch; before dinner; after dinner; and before bed. It should be noted that the actual times or events during/before/after which the device reminds the user to take measurements can vary depending on various factors, such as, for example, the user's habits and lifestyle. It should be noted that the measurement of analyte levels is not continuous and is not automatic. Rather, the device instructs the user to take measurements at several selected discrete times throughout the user's wake hours. As such, this aspect of the present invention allows for a more efficient method of monitoring a user's analyte levels. It should be noted that measurements can be taken more frequently (i.e. more than 6-8 times per day) when more intensive management is required. In one embodiment, in the context of intensive blood-glucose management, blood-glucose is measured frequently at regular time intervals. In another embodiment, the blood-glucose is measured more is measured frequently at non-regular time intervals. For example, more measurements can be taken during wake hours than during other hours in the day.

In yet another aspect of the present invention, the AST device determines when to remind the user to take analyte measurements by taking into consideration the current analyte level, the targeted analyte level, the measured analyte highs and lows, etc. The AST device builds a case history about the user's analyte levels during his/her wake hours and adjusts the times at which it reminds the user to take an analyte measurement. In one embodiment, the AST device utilizes fuzzy logic and/or other predictive analysis methods known in the art to determine when it should remind the user to take analyte measurements. This can require storing and processing large amounts of data. As such, in one embodiment, the data is transmitted to an outside processing circuit, unit, computer, storage device, etc. The processed information is then transmitted back to the AST device. In another embodiment, the AST device also utilizes fuzzy logic and/or other predictive analysis methods known in the art to determine what the target analyte level should be. This target value is then provided to the physician who can consider the suggested target value when making the decision as to what the user's target analyte concentration should be.

In addition to suggesting target values to the physician, the AST device measurements assist physicians in treating patients in any number of ways. In one embodiment, the blood-glucose measurements provided by the AST device can be used to expedite and/or supplement hospital protocols for monitoring and/or treating patients. For example, in a situation where a patient at a hospital is experiencing a hyperglycemic episode, such as, for example a blood-glucose level equal to or exceeding 520 mg/dL, it is common hospital protocol to send draw blood from the patient and send the blood sample out to a laboratory for blood work. One reason for this is that typical methods of blood-glucose measurement, such as, for example, fingerstick measurements, do not provide accurate measurements when the blood-glucose is as high as 520 mg/dL. In one embodiment, the AST device blood-glucose measurements are used by physicians to obtain an accurate and timely blood-glucose value, thereby providing an alternative to typical methods of measuring blood-glucose, and thereby alleviating the need to send blood samples for lab work or to wait for results. In another embodiment, the AST device essentially serves as a portable medical record providing information, including but not limited to analyte and time data values, with which physicians and hospitals can provide improved treatments for patients.

It is to be understood that the patent rights arising hereunder are not to be limited to the specific embodiments or methods described in this specification or illustrated in the drawings, but extend to other arrangements, technology, and methods, now existing or hereinafter arising, which are suitable or sufficient for achieving the purposes and advantages hereof.

What is claimed is:

1. A method for improving the accuracy of analyte concentration measurements, the method comprising:
determining a rate of change of analyte concentration;
measuring an analyte concentration at an alternative site; and
adjusting the measured analyte concentration based on said rate of change to generate an adjusted analyte concentration value which is a more accurate estimate of the systemic analyte concentration value.

2. The method of claim 1, further comprising:
determining the clinical significance of an error in the measured analyte concentration.

3. The method of claim 1, wherein adjusting the measured analyte concentration comprises:
determining a time lag between analyte concentrations measured on-site and at an alternative site;
determining a correction factor based on the time lag and said rate of change of analyte concentration; and
adjusting the measured analyte concentration value by said correction factor.

4. The method of claim 1, further comprising:
correcting the measured analyte concentration based on the rate of change of the analyte concentration, further comprising:
determining the time lag between analyte concentrations measured onsite and at an alternative site;
taking a follow-up analyte concentration measurement at a time that falls between the time of previous analyte concentration measurement and the sum of the time lag and the time of the previous analyte concentration measurement;
determining the rate of analyte concentration change during the time period between the previous analyte concentration measurement and the follow-up analyte concentration measurement; and
extrapolating the analyte concentration value to a time after the sum of the time lag and the time of the previous analyte concentration measurement by using the rate of analyte concentration change in between the previous analyte concentration measurement and the follow-up analyte concentration measurement.

5. The method of claim 1, further comprising:
screening measured analyte concentration values to remove outlier concentration values and/or rate of concentration change values based on statistical analysis, data history, and/or data forecasts or trends.

6. A method for improving the accuracy of information provided to a user by an analyte detection system, said method comprising:
determining a rate of change of analyte concentration; and
adjusting said information provided to said user based on said rate of change.

7. The method of claim 6, wherein determining said rate of change comprises estimating that the absolute value of said rate of change is relatively high.

8. The method of claim 6, wherein determining said rate of change comprises estimating that the absolute value of said rate of change will remain relatively high for about 1-2 hours.

9. The method of claim 6, wherein determining said rate of change comprises receiving data input from the user and estimating, based on said data, that the absolute value of said rate of change is relatively high.

10. The method of claim 9, wherein said data comprises at least one of (i) that the user is about to eat or has just eaten a meal and (ii) that the user is about to engage in or has just engaged in physical exercise.

11. The method of claim 6, wherein determining said rate of change comprises:
measuring a first analyte concentration at a first measurement time;
measuring a second analyte concentration at a second measurement time; and
computing said rate of change based on said first and second analyte concentrations and said first and second measurement times.

12. The method of claim 11, wherein determining said rate of change further comprises continuously computing said rate of change.

13. The method of claim 6, wherein adjusting said information comprises warning the user that the absolute value of said rate of change is relatively high.

14. The method of claim 13, wherein adjusting said information further comprises prompting the user to take an on-site analyte concentration measurement.

15. The method of claim 6, wherein adjusting said information comprises warning the user, when the absolute value of said rate of change is relatively high, that alternative-site measurements are likely to be inaccurate.

16. The method of claim 15, wherein adjusting said information further comprises prompting the user to take an on-site analyte concentration measurement.

17. The method of claim 6, wherein adjusting said information comprises at least one of (i) warning the user that said analyte concentration is increasing and (ii) warning the user that said analyte concentration is decreasing.

18. The method of claim 6, wherein adjusting said information comprises adjusting an analyte concentration measurement based on said rate of change.

19. The method of claim 6, wherein adjusting said information comprises:
measuring a raw analyte concentration;
calculating a corrected analyte concentration based on said rate of change and said raw analyte concentration;
assessing the clinical significance of a measurement error based on said raw analyte concentration and said corrected analyte concentration; and
warning the user to take an on-site analyte concentration measurement based on the clinical significance of said measurement error.

20. The method of claim 19, wherein assessing the clinical significance comprises using an external model.

21. The method of claim 20, wherein the external model comprises a variation of the Clarke error grid.

22. The method of claim 6, wherein determining said rate of change comprises rejecting outlier analyte-concentration measurements.

23. A system for measuring the concentration of an analyte at an alternative site, said system comprising:
a processing circuit; and
a module executable by the processing circuit whereby the processing circuit receives an analyte concentration measurement taken at the alternative site, determines a rate of change of the concentration of said analyte, and provides information to the user based on said rate of change.

24. The system of claim 23, wherein providing information comprises warning the user that the absolute value of the rate of change is high.

25. The system of claim 23, wherein providing information comprises prompting the user to take an on-site analyte concentration measurement.

26. The system of claim 23, wherein providing information comprises warning the user, when the absolute value of said rate of change is relatively high, that alternative-site measurements are likely to be inaccurate.

27. The system of claim 26, wherein providing information further comprises prompting the user to take an on-site analyte concentration measurement.

28. The system of claim 23, wherein providing information comprises at least one of (i) warning the user that the concentration of said analyte is increasing and (ii) warning the user that the concentration of said analyte is decreasing.

29. The system of claim 23, wherein the analyte concentration measurement is a measurement of the concentration of glucose within blood.

30. The system of claim 23, further comprising a timing circuit accessible by said processing circuit.

31. The system of claim 30, wherein the timing circuit comprises a real-time clock.

32. The system of claim 31, wherein the real-time clock is capable of keeping the time of day, the day of week, the day of month, the month, and/or the year.

33. The system of claim 23, further comprising a user-interface whereby the user is able to input data into the analyte detection system, wherein the processing circuit considers said data in adjusting the analyte concentration to generate an adjusted analyte concentration value.

34. The system of claim 33, wherein said data comprises at least one of (i) that the user is about to eat or has just eaten a meal and (ii) that the user is about to engage in or has just engaged in physical exercise.

35. The system of claim 23, wherein the processing circuit assesses the clinical significance of a possible error in said analyte concentration measurement.

36. The system of claim 35, wherein assessing the clinical significance comprises using an external model.

37. The system of claim 36, wherein the external model comprises a variation of the Clarke error grid.

38. The system of claim 23, wherein the processing circuit screens measured analyte concentration values to remove outlier concentration values and/or rate of concentration change values based on statistical analysis, data history, and/or data forecasts or trends.

39. A system for measuring the concentration of an analyte at an alternative site, said system comprising:
a processing circuit; and
a module executable by the processing circuit whereby the processing circuit receives an analyte concentration measurement taken at the alternative site, determines a rate of change of the concentration of said analyte, and adjusts said analyte concentration measurement based on said rate of change.

40. The system of claim 39, wherein the analyte concentration measurement is a measurement of the concentration of glucose within blood.

41. The system of claim 39, further comprising a timing circuit accessible by said processing circuit.

42. The system of claim 41, wherein the timing circuit comprises a real-time clock.

43. The system of claim 42, wherein the real-time clock is capable of keeping the time of day, the day of week, the day of month, the month, and/or the year.

44. The system of claim 39, further comprising a user-interface whereby the user is able to input data into the analyte detection system, wherein the processing circuit considers said data in adjusting the analyte concentration to generate an adjusted analyte concentration value.

45. The system of claim 44, wherein said data comprises at least one of (i) that the user is about to eat or has just eaten a meal and (ii) that the user is about to engage in or has just engaged in physical exercise.

46. The system of claim 39, wherein the processing circuit assesses the clinical significance of a possible error in said analyte concentration measurement.

47. The system of claim 46, wherein assessing the clinical significance comprises using an external model.

48. The system of claim 47, wherein the external model comprises a variation of the Clarke error grid.

49. The system of claim 39, wherein the processing circuit screens measured analyte concentration values to remove outlier concentration values and/or rate of concentration change values based on statistical analysis, data history, and/or data forecasts or trends.
